(19) United States
(12) Patent Application Publication
(10) Pub. No.: US 2009/0105072 A1
(43) Pub. Date: Apr. 23, 2009

(54) 2-(Pyridin-2-Yl)-Pyrimidines for Use as Fungicides

(76) Inventors: Wassilios Grammenos, Ludwigshafen (DE); Thomas Grote, Wachenheim (DE); Jochen Dietz, Mannheim (DE); Jan Klaas Lohmann, Mannheim (DE); Jens Renner, Bad Durkheim (DE); Bernd Muller, Frankenthal (DE); Sarah Ulmschneider, Bad Durkheim (DE)

Correspondence Address: BIRCH STEWART KOLASCH & BIRCH, PO BOX 747, FALLS CHURCH, VA, 22040-0747, US

(21) Appl. No.: 12/225,862

(22) PCT Filed: Apr. 11, 2007
(86) PCT No.: PCT/EP2007/053516

§ 371 Date: Oct. 1, 2008

(30) Foreign Application Priority Data
May 21, 2007 (EP) 06007744.3

(51) Int. Cl. A01N 43/54; C07D 239/70; A01P 3/00; C07D 491/052

(52) U.S. Cl. 504/100; 544/253; 544/255; 514/258.1; 514/266.1

(57) ABSTRACT

The present invention relates to 2-(pyridin-2-yl)pyrimidines and their use for controlling harmful fungi, and also to crop protection compositions comprising such compounds as effective component.

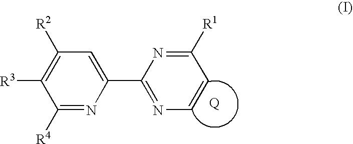

in which:
Q is a fused saturated 5-, 6- or 7-membered carbocycle or a 5-, 6- or 7-membered heterocycle which, in addition to the carbon ring members, has one or two heteroatoms selected from the group consisting of oxygen and sulfur as ring members, where the carbocycle and the heterocycle are unsubstituted or have 1, 2, 3 or 4 C₁-C₄-alkyl groups as substituents;
R¹ is hydrogen, OH, C₁-C₄-alkyl, C₁-C₄-alkoxy, C₁-C₄-haloalkyl, C₁-C₄-haloalkoxy or halogen;
R² is hydrogen, NO₂, halogen, C₁-C₆-alkyl, C₃-C₆-cycloalkyl, C₁-C₆-alkoxy, C₁-C₆-haloalkyl or C₁-C₆-haloalkoxy;
R³ is hydrogen, halogen, C₁-C₄-alkyl, C₁-C₄-alkoxy, C₁-C₄-haloalkyl, or C₁-C₄-haloalkoxy;
R⁴ is phenyl, 5-membered heteroaryl which has 1, 2, 3 or 4 nitrogen atoms or 1 heteroatom selected from the group consisting of oxygen and sulfur and optionally 1, 2 or 3 nitrogen atoms as ring atoms, or 6-membered hetaryl which has 1, 2, 3 or 4 nitrogen atoms as ring members, where phenyl and 5- and 6-membered hetaryl may have 1, 2, 3 or 4 substituents R^{a}.

The present invention relates to 2-(pyridin-2-yl)-pyrimidines and their use for controlling harmful fungi, and also to crop protection compositions comprising such compounds as active component.

EP-A 234 104 describes 2-(pyridin-2-yl)pyrimidines which have an alkyl group in the 6-position of the pyridine radical and which may have a fused 5- or 6-membered ring in the 3,4-position of the pyrimidine ring. The compounds are suitable for controlling phytopathogenic fungi (harmful fungi).

EP-A 259 139 describes 2-(pyridin-2-yl)pyrimidines of the general formula A

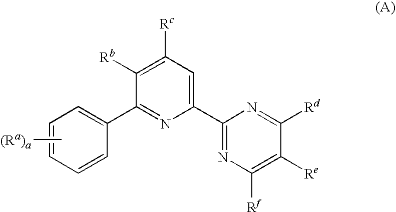

in which a is 0, 1, 2, 3, 4 or 5, R^{a} is halogen, lower alkyl, lower alkoxy or haloalkyl, R^{b} and R^{c} independently of one another are hydrogen or C₁-C₄-alkyl, R^{d} is hydrogen or lower alkyl, R^{e} is hydrogen, lower alkyl or halogen or together with R^{d} is propane-1,3-diyl or butane-1,4-diyl and R^{f} is inter alia hydrogen, alkyl, lower alkoxy or lower alkylthio.

WO 2006/010570 describes fungicidally active 2-(6-phenylpyridin-2-yl)pyrimidine compounds of the formula B below:

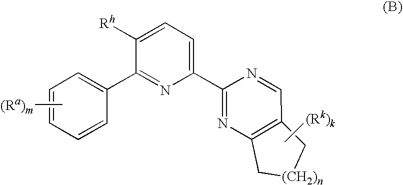

in which: k is 0, 1, 2 or 3, m is 0, 1, 2, 3, 4 or 5 and n is 1, 2, 3, 4 or 5, the substituents R⁹ are inter alia halogen, OH, CN, NO₂, C₁-C₄-alkyl, C₁-C₄-haloalkyl, C₁-C₄-alkoxy, C₁-C₄-haloalkoxy, C₂-C₄-alkenyl, C₂-C₄-alkynyl, C₃-C₈-cycloalkyl, C₁-C₄-alkoxy-C₁-C₄-alkyl, amino, phenoxy, etc., R^{h} is C₁-C₄-haloalkyl, C₁-C₄-alkoxy, C₁-C₄-haloalkoxy, hydroxyl, halogen, CN or NO₂ and R^{k} is C₁-C₄-alkyl.

With respect to their fungicidal activity, some of the 2-(pyridin-2-yl)pyrimidines known from the prior art are unsatisfactory, or they have unwanted properties such as low crop plant compatibility.

Accordingly, it is an object of the present invention to provide novel compounds having improved fungicidal activity and/or better compatibility with crop plants.

Surprisingly, this object is achieved by 2-(pyridin-2-yl)-pyrimidine compounds of the general formula I

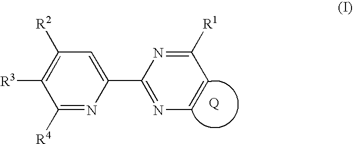

in which:
Q is a fused saturated 5-, 6- or 7-membered carbocycle or a 5-, 6- or 7-membered heterocycle which, in addition to the carbon ring members, has one or two heteroatoms selected from the group consisting of oxygen and sulfur as ring members, where the carbocycle and the heterocycle are unsubstituted or have 1, 2, 3 or 4 C₁-C₄-alkyl groups as substituents;
R¹ is hydrogen, OH, C₁-C₄-alkyl, C₁-C₄-alkoxy, C₁-C₄-haloalkyl, C₁-C₄-haloalkoxy or halogen;
R² is hydrogen, NO₂, halogen, C₁-C₆-alkyl, C₃-C₆-cycloalkyl, C₁-C₆-alkoxy, C₁-C₆-haloalkyl or C₁-C₆-haloalkoxy;
R³ is hydrogen, halogen, C₁-C₄-alkyl, C₁-C₄-alkoxy, C₁-C₄-haloalkyl or C₁-C₄-haloalkoxy;
R⁴ is phenyl, 5-membered heteroaryl which has 1, 2, 3 or 4 nitrogen atoms or 1 heteroatom selected from the group consisting of oxygen and sulfur and optionally 1, 2 or 3 nitrogen atoms as ring atoms, or 6-membered hetaryl which has 1, 2, 3 or 4 nitrogen atoms as ring members, where phenyl and 5- and 6-membered hetaryl may have 1, 2, 3 or 4 substituents R^{a}, where
R^{a} is selected from the group consisting of OH, SH, halogen, NO₂, NH₂, CN, COOH, CONH₂, C₁-C₈-alkyl, C₁-C₈-alkoxy, C₁-C₈-haloalkyl, C₁-C₈-haloalkoxy, C₁-C₈-alkylamino, di(C₁-C₈-alkyl)amino, C₁-C₈-alkylthio, C₁-C₈-haloalkylthio, C₁-C₈-alkylsulfinyl, C₁-C₈-haloalkylsulfinyl, C₁-C₈-alkylsulfonyl, C₁-C₈-haloalkylsulfonyl, C₃-C₈-cycloalkyl, phenyl, phenoxy and radicals of the formula C(=Z)R^{aa} in which Z is O, S, N(C₁-C₈-alkyl), N(C₁-C₈-alkoxy), N(C₃-C₈-alkenyloxy) or N(C₃-C₈-alkynyloxy) and R^{aa} is hydrogen, C₁-C₄-alkyl, C₁-C₄-alkoxy, NH₂, C₁-C₈-alkylamino or di(C₁-C₈-alkyl)amino;
   and the agriculturally useful salts of the compounds of the formula I;
   except for compounds of the formula I in which R² is hydrogen or C₁-C₆-alkyl, R⁴ is phenyl which optionally carries 1, 2, 3 or 4 substituents R^{a} and Q is a fused saturated 5-, 6- or 7-membered carbocycle which is unsubstituted or has 1, 2, 3 or 4 C₁-C₄-alkyl groups as substituents and the agriculturally useful salts of these compounds.

Accordingly, the present invention provides the 2-(pyridin-2-yl)pyrimidines of the general formula I and their agriculturally acceptable salts.

The present invention furthermore provides the use of the 2-(pyridin-2-yl)pyrimidines of the general formula I and their agriculturally acceptable salts for controlling phytopathogenic fungi (=harmful fungi), and also a method for controlling phytopathogenic fungi wherein the fungi or the materials, plants, the soil or seed to be protected against fungal attack are/is treated with an effective amount of a compound of the general formula I and/or with an agriculturally acceptable salt of 1.

The present invention furthermore provides a composition for controlling harmful fungi and comprising at least one 2-(pyridin-2-yl)pyrimidine compound of the general formula I and/or an agriculturally acceptable salt thereof and at least one liquid or solid carrier.

Depending on the substitution pattern, the compounds of the formula I and their tautomers may have one or more centers of chirality, in which case they are present as pure enantiomers or pure diastereomers or as enantiomer or diastereomer mixtures. The invention provides both the pure enantiomers or diastereomers and their mixtures.

Agriculturally useful salts encompass especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting I with an acid of the corresponding anion, preferably of hydrochloric acid, hydrobromic acid, sulfuric acid, phosphoric acid or nitric acid.

In the definitions of the variables given in the formulae above, collective terms are used which are generally representative for the substituents in question. The term Cₙ-Cₘ indicates the number of carbon atoms possible in each case in the substituent or substituent moiety in question:

halogen: fluorine, chlorine, bromine and iodine;
alkyl and also all alkyl moieties in alkoxy, alkoxyalkyl, alkylcarbonyl, alkoxycarbonyl, alkylthio, alkylsulfonyl, alkylsulfinyl, alkylamino, dialkylamino, alkylaminocarbonyl, dialkylaminocarbonyl: saturated, straight-chain or branched hydrocarbon radicals having 1 to 8 (C₁-C₈-alkyl), frequently 1 to 6 (C₁-C₆-alkyl) and in particular 1 to 4 carbon atoms (C₁-C₄-alkyl), such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, heptyl, 1-methylhexyl, octyl, 1-methylheptyl and 2-ethylhexyl;
haloalkyl and also all haloalkyl moieties in haloalkoxy and haloalkylthio: straight-chain or branched alkyl groups having 1 to 8 and in particular 1 to 4 carbon atoms (as mentioned above), where some or all of the hydrogen atoms in these groups may be replaced by halogen atoms as mentioned above, in particular fluorine and chlorine: in particular C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl;
alkenyl: monounsaturated, straight-chain or branched hydrocarbon radicals having 2 to 8 or 3 to 8 carbon atoms and a double bond in any position, for example ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl;
alkynyl: straight-chain or branched hydrocarbon groups having 2 to 8 or 3 to 8 carbon atoms and a triple bond in any position, for example ethynyl, 1-propynyl, 2-propynyl, 1-butynyl, 2-butynyl, 3-butynyl, 1-methyl-2-propynyl;
cycloalkyl: monocyclic saturated hydrocarbon groups having 3 to 8, preferably to 6, carbon ring members, such as cyclopropyl, cyclobutyl, cyclopentyl and cyclohexyl;
cycloalkylmethyl: a cycloalkyl radical as mentioned above which is attached via a methylene group (CH₂); alkylamino and also the alkylamino moieties in alkylaminocarbonyl: an alkyl group which is attached via an NH group, where alkyl is one of the alkyl radicals mentioned above having 1 to 8 carbon atoms, such as methylamino, ethylamino, n-propylamino, isopropylamino, n-butylamino and the like;
dialkylamino and also the dialkylamino moieties in dialkylaminocarbonyl: a radical of the formula N(alkyl)₂, where alkyl is one of the alkyl radical mentioned above having 1 to 8 carbon atoms, for example dimethylamino, diethylamino, methylethylamino, N-methyl-N-propylamino and the like;
alkoxy and also the alkoxy moieties in alkoxycarbonyl: an alkyl group, attached via an oxygen, having 1 to 8, in particular 1 to 6 and especially 1 to 4 carbon atoms, for example methoxy, ethoxy, n-propoxy, 1-methylethoxy, butoxy, 1-methylpropoxy, 2-methylpropoxy or 1,1-dimethylethoxy;
alkoxycarbonyl: an alkoxy radical as mentioned above, attached via a carbonyl group;
alkylthio: an alkyl group as mentioned above, attached via a sulfur atom;
alkylsulfinyl: an alkyl group as mentioned above, attached via an S(═O) group;
alkylsulfonyl: an alkyl group as mentioned above, attached via an S(═O)₂ group;
haloalkoxy: an alkoxy radical having 1 to 8, in particular 1 to 6 and especially 1 to 4 carbon atoms as mentioned above which is partially or fully substituted by fluorine, chlorine, bromine and/or iodine, preferably substituted by fluorine, i.e., for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy;
alkylene: a straight-chain saturated hydrocarbon chain having 2 to 6 and in particular 2 to 4 carbon atoms, such as ethane-1,2-diyl, propane-1,3-diyl, butane-1,4-diyl, pentane-1,5-diyl or hexane-1,6-diyl.

Saturated 5-, 6- or 7-membered heterocycle which has one or two heteroatoms selected from the group consisting of oxygen and sulfur as ring members: a ring constructed of carbon atoms and 1 or 2 heteroatoms selected from the group consisting of oxygen and sulfur, the total number of ring atoms (ring members) being 5, 6 or 7, for example: oxolane, oxepane, oxane (hexahydropyran), 1,3-dioxolane, 1,3-dioxane, 1,4-dioxane, thiolane, thiane, thiepane, 1,3-dithiolane, 1,3-dithiane and 1,4-dithiane;

5- or 6-membered heteroaryl: a 5- or 6-membered aromatic ring which, in addition to carbon, has 1, 2, 3 or 4 heteroatoms as ring members, the heteroatoms typically being selected from the group consisting of oxygen, nitrogen and sulfur, in particular:
5-membered heteroaryl which has 1, 2, 3 or 4 nitrogen atoms as ring members, such as 1-, 2- or 3-pyrrolyl, 1-, 3- or 4-pyrazolyl, 1-, 3- or 4-imidazolyl, 1,2,3-[1H]-triazol-1-yl, 1,2,3-[2H]-triazol-2-yl, 1,2,3-[1H]-triazol-4-yl, 1,2,3-[1H]-triazol-5-yl, 1,2,3-[2H]-triazol-4-yl, 1,2,4-[1H]-triazol-1-yl, 1,2,4-[1H]-triazol-3-yl, 1,2,4-[1H]-triazol-5-yl, 1,2,4-[4H]-triazol-4-yl, 1,2,4-[4H]-triazol-3-yl, [1H]-tetrazol-1-yl, [1H]-tetrazol-5-yl, [2H]-tetrazol-2-yl and [2H]-tetrazol-5-yl;
5-membered heteroaryl which has 1 heteroatom selected from the group consisting of oxygen and sulfur and optionally 1, 2 or 3 nitrogen atoms as ring members, for example 2-furyl, 3-furyl, 2-thienyl, 3-thienyl, 3- or 4-isoxazolyl, 3- or 4-isothiazolyl, 2-, 4- or 5-oxazolyl, 2-, 4 or 5-thiazolyl, 1,2,4-thiadiazol-3-yl, 1,2,4-thiadiazol-5-yl, 1,3,4-thiadiazol-2-yl, 1,2,4-oxadiazol-3-yl, 1,2,4-oxadiazol-5-yl and 1,3,4-oxadiazol-2-yl;
6-membered heteroaryl which has 1, 2, 3 or 4 nitrogen atoms as ring members, such as 2-pyridinyl, 3-pyridinyl, 4-pyridinyl, 2-pyrimidinyl, 4-pyrimidinyl, 5-pyrimidinyl, 2-pyrazinyl, 3-pyridazinyl, 4-pyridazinyl, 1,2,4-triazin-3-yl, 1,2,4-triazin-5-yl, 1,2,4-triazin-6-yl and 1,3,5-triazinyl.

With a view to the use as fungicides, preference is given to those compounds of the formula I in which the variables Q, R¹, R², R³ and R⁴ independently of one another and in particular in combination have the following meanings:

According to the invention, Q together with the carbon atoms of the 4- and the 5-position of the pyrimidine ring to which Q is attached are a saturated 5-, 6- or 7-membered carbocycle or heterocycle which is as defined above and may carry one or more C₁-C₄-alkyl groups as substituents. In particular Q together with the carbon atoms of the pyrimidine ring to which it is attached is one of the rings below:

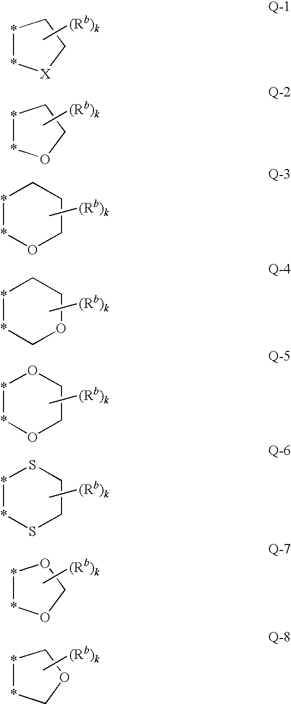

in which
* are the atoms of the pyrimidine ring;
k is 0, 1, 2, 3 or 4;
R^{b} is C₁-C₄-alkyl, in particular methyl; and
X is (CH₂)ₙ where n=1, 2 or 3.

The radicals R^{b} can be located at any carbon atoms of these rings, and 1, 2, 3 or 4 of the hydrogen atoms in (CH₂)ₙ may be replaced by R^{b}, for example if k≠0. The radicals Q-2, Q-3 and Q-4 can assume any orientation with respect to the pyrimidine ring. From among the radicals Q-1 to Q-8, particular preference is given to the radicals Q-1 and Q-3 and especially to radicals Q-1 where n=2 or 3. The variable k is in particular 0, 1 or 2.

R¹ is preferably selected from the group consisting of hydrogen, fluorine, chlorine, methyl, ethyl, methoxy, ethoxy, CF₃, CHF₂, OCF₃ and OCHF₂. With particular preference, R¹ is hydrogen.

R² is preferably selected from the group consisting of hydrogen, fluorine, chlorine, C₁-C₄-alkyl, especially methyl, ethyl, isopropyl or tert-butyl, methoxy, CF₃, CHF₂, OCF₃ and OCHF₂. Particular preference is given to compounds of the formula I in which R² is hydrogen. Particular preference is likewise given to compounds of the formula I in which R² is methyl. Particular preference is likewise given to compounds of the formula I in which R² is methoxy. Particular preference is likewise given to compounds of the formula I in which R² is chlorine.

R³ is preferably a radical different from hydrogen. From among these, particular preference is given to compounds of the formula I in which R³ is fluorine, chlorine, C₁-C₄-alkyl, especially methyl, or methoxy. Very particularly preferably, R³ is methyl, fluorine or methoxy.

With respect to their fungicidal action, preference is given to compounds of the general formula I in which R⁴ is one of the radicals below:
5-membered heteroaryl which, in addition to carbon, has 1, 2, 3 or 4 nitrogen atoms as ring atoms;
5-membered heteroaryl which, in addition to carbon, has 1 heteroatom selected from the group consisting of oxygen and sulfur and optionally 1, 2 or 3 nitrogen atoms as ring atoms, in particular thienyl or furyl;
6-membered hetaryl which has 1, 2, 3 or 4 nitrogen atoms as ring atoms, in particular pyridyl or pyrimidinyl;
   where 5- and 6-membered hetaryl may be unsubstituted or some or all of the hydrogen atoms in the unsubstituted hetaryl may be replaced by substituents R^{a} of the type indicated above, so that the total number of all substituents R^{a} on hetaryl is typically 1, 2, 3 or 4. Substituents on nitrogen ring atoms are in particular R^{a} attached via carbon and especially C₁-C₄-alkyl.

Preferred radicals R^{a} are selected from the group consisting of halogen, C₁-C₄-alkyl, C₁-C₂-haloalkyl, C₁-C₄-alkoxy, C₁-C₂-haloalkoxy, C₁-C₄-alkylthio, C₁-C₄-alkylcarbonyl, C₁-C₄-alkoxycarbonyl, and radicals of the formula C(═N—O—C₁-C₈-alkyl)R^{aa} in which R^{aa} is hydrogen or C₁-C₄-alkyl. Especially preferably, the radicals R^{a} are selected from the group consisting of halogen, especially chlorine or fluorine, methyl, methoxy, trifluoromethyl, difluoromethyl, trifluoromethoxy, difluoromethoxy and methylthio.

In this embodiment, R⁴ is preferably optionally substituted 2-furyl, 3-furyl, 2-thienyl, 3-thienyl, 2-pyridyl, 3-pyridyl, 4-pyridyl, 2-pyrimidinyl, 4-pyrimidinyl or 5-pyrimidinyl, where the heterocyclic radicals mentioned above are preferably unsubstituted or have 1, 2 or 3 substituents R^{a}. With respect to the preferred and particularly preferred radicals, what has been said above applies.

From among the heteroaromatic radicals R⁴, particular preference is given to those radicals which have at least one substituent and/or at least one ring member selected from the group consisting of O, S and N as ring member in the ortho-position to the point of attachment of R⁴ to the pyridine ring.

Examples of preferred heteroaromatic radicals R⁴ are
optionally substituted 2-thienyl, such as unsubstituted 2-thienyl, 5-methylthiophen-2-yl, 4-methylthiophen-2-yl, 5-chlorothiophen-2-yl, 3-cyanothiophen-2-yl, 4-bromothiophen-2-yl, 3,5-dichlorothiophen-2-yl, 3,4,5-trichlorothiophen-2-yl, 5-bromothiophen-2-yl,
optionally substituted 3-thienyl, such as unsubstituted 3-thienyl, 2-methylthiophen-3-yl, 2,5-dichlorothiophen-3-yl,
optionally substituted 2-furyl, such as unsubstituted 2-furyl, 5-methylfuran-2-yl, 5-chlorofuran-2-yl, 4-methylfuran-2-yl, 3-cyanofuran-2-yl, 5-acetylfuran-2-yl, 5-bromofuran-2-yl, 3,5-dichlorofuran-2-yl,
optionally substituted 3-furyl, such as unsubstituted 3-furyl, 2-methylfuran-3-yl, 2,5-dimethylfuran-3-yl,
optionally substituted 2-pyridyl, such as unsubstituted 2-pyridyl, 3-fluoropyridin-2-yl, 3-chloropyridin-2-yl, 3-bromopyridin-2-yl, 3-trifluoromethyl-pyridin-2-yl, 3-methylpyridin-2-yl, 3-ethylpyridin-2-yl, 3,5-difluoropyridin-2-yl, 3,5-dichloropyridin-2-yl, 3,5-dibromopyridin-2-yl, 3,5-dimethylpyridin-2-yl, 3-fluoro-5-trifluoromethylpyridin-2-yl, 3-chloro-5-fluoropyridin-2-yl, 3-chloro-5-methylpyridin-2-yl, 3-fluoro-5-chloropyridin-2-yl, 3-fluoro-5-methylpyridin-2-yl, 3-methyl-5-fluoropyridin-2-yl, 3-methyl-5-chloropyridin-2-yl, 5-nitropyridin-2-yl, 5-cyanopyridin-2-yl, 5-methoxycarbonylpyridin-2-yl, 5-trifluoromethylpyridin-2-yl, 5-methylpyridin-2-yl, 4-methylpyridin-2-yl, 6-methylpyridin-2-yl,
optionally substituted 3-pyridyl, such as unsubstituted 3-pyridyl, 2-chloropyridin-3-yl, 2-bromopyridin-3-yl, 2-methylpyridin-3-yl, 2,4-dichloropyridin-3-yl, 2,4-dibromopyridin-3-yl, 2,4-difluoropyridin-3-yl, 2-fluoro-4-chloropyridin-3-yl, 2-chloro-4-fluoropyridin-3-yl, 2-chloro-4-methylpyridin-3-yl, 2-methyl-4-fluoropyridin-3-yl, 2-methyl-4-chloropyridin-3-yl, 2,4-dimethylpyridin-3-yl, 2,4,6-trichloropyridin-3-yl, 2,4,6-tribromopyridin-3-yl, 2,4,6-trimethylpyridin-3-yl, 2,4-dichloro-6-methylpyridin-3-yl,
optionally substituted 4-pyridyl, such as unsubstituted 4-pyridyl, 3-chloropyridin-4-yl, 3-bromopyridin-4-yl, 3-methylpyridin-4-yl, 3,5-dichloropyridin-4-yl, 3,5-dibromo-pyridin-4-yl, 3,5-dimethylpyridin-4-yl,
optionally substituted 4-pyrimidinyl, such as unsubstituted 4-pyrimidinyl, 5-chloropyrimidin-4-yl, 5-fluoropyrimidin-4-yl, 5-fluoro-6-chloropyrimidin-4-yl, 2-methyl-6-trifluoromethylpyrimidin-4-yl, 2,5-dimethyl-6-trifluoromethylpyrimidin-4-yl, 5-methyl-6-trifluoromethyl-pyrimidin-4-yl, 6-trifluoromethylpyrimidin-4-yl, 2-methyl-5-fluoropyrimidin-4-yl, 2-methyl-5-chloropyrimidin-4-yl, 5-chloro-6-methyl-pyrimidin-4-yl, 5-chloro-6-ethylpyrimidin-4-yl, 5-chloro-6-isopropylpyrimidin-4-yl, 5-bromo-6-methylpyrimidin-4-yl, 5-fluoro-6-methylpyrimidin-4-yl, 5-fluoro-6-fluoromethylpyrimidin-4-yl, 2,6-dimethyl-5-chloropyrimidin-4-yl, 5,6-dimethyl-pyrimidin-4-yl, 2,5-dimethylpyrimidin-4-yl, 2,5,6-trimethylpyrimidin-4-yl, 5-methyl-6-methoxypyrimidin-4-yl,
optionally substituted 5-pyrimidinyl, such as unsubstituted 5-pyrimidinyl, 4-methyl-pyrimidin-5-yl, 4,6-dimethylpyrimidin-5-yl, 2,4,6-trimethylpyrimidin-5-yl, 4-trifluoromethyl-6-methylpyrimidin-5-yl,
optionally substituted 2-pyrimidinyl, such as unsubstituted 2-pyrimidinyl, 4,6-dimethylpyrimidin-2-yl, 4,5,6-trimethylpyrimidin-2-yl, 4,6-ditrifluoromethylpyrimidin-2-yl and 4,6-dimethyl-5-chloropyrimidin-2-yl.

According to another preferred embodiment, R⁴ is optionally substituted phenyl. If R⁴ is optionally substituted phenyl, Q is preferably a 5-, 6- or 7-membered heterocycle which is as defined above and which may carry one or more C₁-C₄-alkyl groups as substituents. In this case, Q is in particular one of the radicals Q-2, Q-3, Q-4, Q-5, Q-6, Q-7 or Q-8 and especially one of the radicals Q-2, Q-3 or Q-4.

If R⁴ is phenyl which is optionally substituted by 1, 2, 3 or 4 radicals R^{a} and R² is different from hydrogen and C₁-C₆-alkyl, Q may also be a 5-, 6- or 7-membered carbocycle which is as defined above and which may carry one or more C₁-C₄-alkyl groups as substituents. In this case, Q is preferably a radical Q-1 where n=2 or 3. The variable k is in particular 0, 1 or 2. In this case, R² is in particular fluorine, chlorine, methoxy, CF₃, CHF₂, OCF₃ or OCHF₂, especially methoxy or chlorine.

In this embodiment, R⁴ is preferably a radical of the formula P:

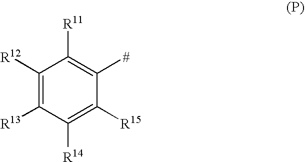

in which # is the point of attachment to the pyridine ring and R¹¹, R¹², R¹³, R¹⁴ and R¹⁵ are hydrogen or at least one of these radicals, for example 1, 2, 3, 4 or 5 of these radicals, has/have one of the meanings given for R^{a}, in particular one of the meanings given as being preferred or particularly preferred. In a preferred embodiment, at least one and especially 1, 2 or 3 of the radicals R¹¹, R¹², R¹³, R¹⁴ or R¹⁵ is/are different from hydrogen. In particular:
R¹¹ is hydrogen, fluorine, chlorine, CH₃, OCH₃, OCHF₂, OCF₃ or CF₃;
R¹², R¹⁴ independently of one another are hydrogen, chlorine, fluorine, CH₃, OCH₃, OCHF₂, OCF₃ or CF₃, where one of the radicals R¹² and R¹⁴ may also be NO₂, C(O)CH₃ or COOCH₃; in particular, R¹² and R¹⁴ are hydrogen, fluorine, methyl or trifluoromethyl;
R¹³ is hydrogen, fluorine, chlorine, cyano, OH, CHO, NO₂, NH₂, methylamino, dimethylamino, diethylamino, C₁-C₄-alkyl, especially CH₃, C₂H₅, CH(CH₃)₂, C₃-C₈-cycloalkyl, especially cyclopropyl, cyclopentyl or cyclohexyl, C₁-C₄-alkoxy, especially OCH₃, C₁-C₄-alkylthio, especially methylthio or ethylthio, C₁-C₄-haloalkyl, especially CF₃, C₁-C₄-haloalkoxy, especially OCHF₂ or OCF₃, or CO(A²) where A² is C₁-C₄-alkyl, especially methyl, or C₁-C₄-alkoxy, especially OCH₃, or a group C(R^{13a})═NOR^{13b} in which R^{13a} is hydrogen or methyl and R^{13b} is C₁-C₄-alkyl, propargyl or allyl or R¹² and R¹³ together form a group O—CH₂—O; and
R¹⁵ is hydrogen, fluorine, chlorine, or C₁-C₄-alkyl, especially CH₃, in particular hydrogen or fluorine.

Advantageously, if more than one of the radicals R¹¹, R¹², R¹³, R¹⁴ or R¹⁵ is different from hydrogen, then only one of the radicals different from hydrogen is different from halogen or methyl. Especially if one of the radicals R¹¹, R¹², R¹³, R¹⁴ or R¹⁵ is different from hydrogen, halogen or methyl, the remaining radicals R¹¹, R¹², R¹³, R¹⁴, R¹⁵ are selected from the group consisting of halogen and hydrogen.

Examples of radicals P are the radicals mentioned below: phenyl, 2-fluorophenyl, 3-fluorophenyl, 4-fluorophenyl, 2-chlorophenyl, 3-chlorophenyl, 4-chlorophenyl, 3-bromophenyl, 4-bromophenyl, 2-trifluoromethylphenyl, 3-trifluoromethylphenyl, 4-trifluoromethylphenyl, 2-(methylthio)phenyl, 3-(methylthio)phenyl, 4-(methylthio)phenyl, 2-methoxyphenyl, 3-methoxyphenyl, 4-methoxyphenyl, 3-nitrophenyl, 4-nitrophenyl, 4-cyanophenyl, 4-aminocarbonylphenyl, 4-formylphenyl, 4-tert-butylphenyl, 4-isopropylphenyl, 3-ethoxyphenyl, 4-ethoxyphenyl, 4-n-propoxyphenyl, 4-isopropoxyphenyl, 3-isopropoxyphenyl, 4-n-butoxyphenyl, 4-tert-butoxyphenyl, 4-acetylphenyl, 4-methoxycarbonylphenyl, 4-ethoxycarbonylphenyl, 4-tert-butoxy-carbonylphenyl, 4-(methoxyiminomethyl)phenyl, 4-(1-(methoxyimino)ethyl)phenyl, 2,3-difluorophenyl, 2,4-difluorophenyl, 2,5-difluorophenyl, 3,4-difluorophenyl, 3,5-difluorophenyl, 2,6-difluorophenyl, 2,4,6-trifluorophenyl, 2,4,5-trifluorophenyl, 2,3,4-trifluorophenyl, 2,3,5-trifluorophenyl, 3,4,5-trifluorophenyl, 2,3-dichlorophenyl, 2,5-dichlorophenyl, 3,5-dichlorophenyl, 2,6-dichlorophenyl, 2,3-dimethylphenyl, 2,4-dimethylphenyl, 2,5-dimethylphenyl, 2,4,5-trimethylphenyl, 2,3-dimethoxyphenyl, 2,4-dimethoxyphenyl, 3,4-dimethoxyphenyl, 2,4-bis(trifluoromethyl)phenyl, 3,5-bis(trifluoromethyl)phenyl, 2-methyl-3-methoxyphenyl, 2-methyl-4-methoxyphenyl, 2-methyl-6-methoxyphenyl, 3-chloro-4-fluorophenyl, 2-chloro-4-fluorophenyl, 2-chloro-6-fluorophenyl, 4-chloro-2-fluorophenyl, 5-chloro-2-fluorophenyl, 4-fluoro-3-methylphenyl, 2-fluoro-4-methylphenyl, 4-fluoro-2-methylphenyl, 2-fluoro-3-methoxyphenyl, 2-fluoro-4-methoxyphenyl, 2-fluoro-6-methoxyphenyl, 2-fluoro-4-trifluoromethylphenyl, 4-chloro-3-methylphenyl, 2-chloro-4-methylphenyl, 2-chloro-6-methylphenyl, 3-chloro-2-methylphenyl, 5-chloro-2-methylphenyl, 2-chloro-4-methoxyphenyl, 2-chloro-6-methoxyphenyl, 2-chloro-4-trifluoromethylphenyl, 3-fluoro-4-methylphenyl, 4-fluoro-3-methylphenyl, 3-fluoro-4-methoxyphenyl, 3-fluoro-4-ethoxyphenyl, 3-fluoro-4-trifluoromethylphenyl, 3-chloro-4-methylphenyl, 3-chloro-4-methoxyphenyl, 3-chloro-4-ethoxyphenyl, 3-chloro-4-trifluoromethylphenyl, 3-methyl-4-methoxyphenyl, 4-chloro-2,5-difluorophenyl, 2-fluoro-4-formylphenyl, 4-tert-butyl-2-fluorophenyl, 2-fluoro-4-isopropylphenyl, 4-ethoxy-2-fluorophenyl, 4-acetyl-2-fluorophenyl, 4-methoxycarbonyl-2-fluorophenyl, 4-ethoxycarbonyl-2-fluorophenyl, 4-tert-butoxycarbonyl-2-fluorophenyl.

Especially preferred are the following groups of compounds of the formula I:

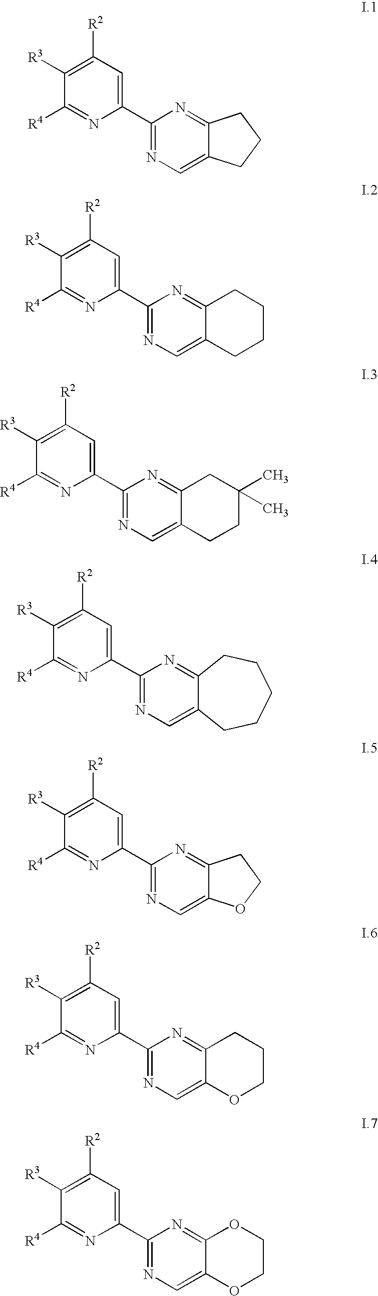

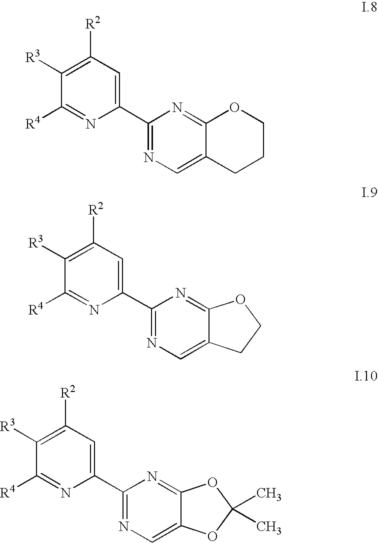

In particular with a view to their use, preference is given to the compounds I compiled in Tables 1 to 16 below.

### Table 1

Compounds of the formulae I.1, I.2, I.3 and I.4 in which R² is hydrogen and the combination of R³ and R⁴ for a compound corresponds in each case to one of rows A-331 to A-638 of Table A.

### Table 2

Compounds of the formulae I.1, I.2, I.3 and I.4 in which R² is methyl and the combination of R³ and R⁴ for a compound corresponds in each case to one of rows A-331 to A-638 of Table A.

### Table 3

Compounds of the formulae I.1, I.2, I.3 and I.4 in which R² is isopropyl (CH(CH₃)₂) and the combination of R³ and R⁴ for a compound corresponds in each case to one of rows A-331 to A-638 of Table A.

### Table 4

Compounds of the formulae I.1, I.2, I.3 and I.4 in which R² is tert-butyl (C(CH₃)₃) and the combination of R³ and R⁴ for a compound corresponds in each case to one of rows A-331 to A-638 of Table A.

### Table 5

Compounds of the formulae I.1, I.2, I.3 and I.4 in which R² is methoxy and the combination of R³ and R⁴ for a compound corresponds in each case to one of the rows of Table A.

### Table 6

Compounds of the formulae I.1, I.2, I.3 and I.4 in which R² is fluorine and the combination of R³ and R⁴ for a compound corresponds in each case to one of the rows of Table A.

### Table 7

Compounds of the formulae I.1, I.2, I.3 and I.4 in which R² is chlorine and the combination of R³ and R⁴ for a compound corresponds in each case to one of the rows of Table A.

### Table 8

Compounds of the formulae I.1, I.2, I.3 and I.4 in which R² is trifluoromethyl and the combination of R³ and R⁴ for a compound corresponds in each case to one of the rows of Table A.

### Table 9

Compounds of the formulae I.5, I.6, I.7, I.8, I.9 and I.10 in which R² is hydrogen and the combination of R³ and R⁴ for a compound corresponds in each case to one of the rows of Table A.

### Table 10

Compounds of the formulae I.5, I.6, I.7, I.8, I.9 and I.10 in which R² is methyl and the combination of R³ and R⁴ for a compound corresponds in each case to one of the rows of Table A.

### Table 11

Compounds of the formulae I.5, I.6, I.7, I.8, I.9 and I.10 in which R² is isopropyl (CH(CH₃)₂) and the combination of R³ and R⁴ for a compound corresponds in each case to one of the rows of Table A.

### Table 12

Compounds of the formulae I.5, I.6, I.7, I.8, I.9 and I.10 in which R² is tert-butyl (C(CH₃)₃) and the combination of R³ and R⁴ for a compound corresponds in each case to one of the rows of Table A.

### Table 13

Compounds of the formulae I.5, I.6, I.7, I.8, I.9 and I.10 in which R² is methoxy and the combination of R³ and R⁴ for a compound corresponds in each case to one of the rows of Table A.

### Table 14

Compounds of the formulae I.5, I.6, I.7, I.8, I.9 and I.10 in which R² is fluorine and the combination of R³ and R⁴ for a compound corresponds in each case to one of the rows of Table A.

### Table 15

Compounds of the formulae I.5, I.6, I.7, I.8, I.9 and I.10 in which R² is chlorine and the combination of R³ and R⁴ for a compound corresponds in each case to one of the rows of Table A.

### Table 16

Compounds of the formulae I.5, I.6, I.7, I.8, I.9 and I.10 in which R² is trifluoromethyl and the combination of R³ and R⁴ for a compound corresponds in each case to one of the rows of Table A.

TABLE A

| No. | R³ | R⁴ |
|---|---|---|
| A-1 | CH₃ | phenyl |
| A-2 | CH₃ | 2-fluorophenyl |
| A-3 | CH₃ | 3-fluorophenyl |
| A-4 | CH₃ | 4-fluorophenyl |
| A-5 | CH₃ | 2-chlorophenyl |
| A-6 | CH₃ | 3-chlorophenyl |
| A-7 | CH₃ | 4-chlorophenyl |
| A-8 | CH₃ | 2-trifluoromethylphenyl |
| A-9 | CH₃ | 3-trifluoromethylphenyl |
| A-10 | CH₃ | 4-trifluoromethylphenyl |
| A-11 | CH₃ | 2-(methylthio)phenyl |
| A-12 | CH₃ | 3-(methylthio)phenyl |
| A-13 | CH₃ | 4-(methylthio)phenyl |
| A-14 | CH₃ | 2-methoxyphenyl |
| A-15 | CH₃ | 3-methoxyphenyl |
| A-16 | CH₃ | 4-methoxyphenyl |
| A-17 | CH₃ | 3-nitrophenyl |
| A-18 | CH₃ | 4-nitrophenyl |
| A-19 | CH₃ | 4-cyanophenyl |
| A-20 | CH₃ | 4-aminocarbonylphenyl |
| A-21 | CH₃ | 4-formylphenyl |
| A-22 | CH₃ | 4-tert-butylphenyl |
| A-23 | CH₃ | 4-isopropylphenyl |
| A-24 | CH₃ | 4-ethoxyphenyl |
| A-25 | CH₃ | 4-n-propoxyphenyl |
| A-26 | CH₃ | 4-isopropoxyphenyl |
| A-27 | CH₃ | 4-n-butoxyphenyl |
| A-28 | CH₃ | 4-tert-butoxyphenyl |
| A-29 | CH₃ | 4-acetylphenyl |
| A-30 | CH₃ | 4-methoxycarbonylphenyl |
| A-31 | CH₃ | 4-ethoxycarbonylphenyl |
| A-32 | CH₃ | 4-tert-butoxycarbonylphenyl |
| A-33 | CH₃ | 4-(methoxyiminomethyl)phenyl |
| A-34 | CH₃ | 4-(1-(methoxyimino)ethyl)phenyl |
| A-35 | CH₃ | 3-isopropoxyphenyl |
| A-36 | CH₃ | 3-ethoxyphenyl |
| A-37 | CH₃ | 3-bromophenyl |
| A-38 | CH₃ | 4-bromophenyl |
| A-39 | CH₃ | 3,4-dimethoxyphenyl |
| A-40 | CH₃ | 3-fluoro-4-isopropylphenyl |
| A-41 | CH₃ | 3-chloro-4-isopropylphenyl |
| A-42 | CH₃ | 2,3,5-trifluorophenyl |
| A-43 | CH₃ | 2,4,5-trifluorophenyl |
| A-44 | CH₃ | 2,3,4-trifluorophenyl |
| A-45 | CH₃ | 2,4,5-trimethylphenyl |
| A-46 | CH₃ | 2-fluoro-3-methoxyphenyl |
| A-47 | CH₃ | 2-fluoro-6-methoxyphenyl |
| A-48 | CH₃ | 5-chloro-2-methylphenyl |
| A-49 | CH₃ | 3-chloro-2-methylphenyl |
| A-50 | CH₃ | 2-fluoro-5-chlorphenyl |
| A-51 | CH₃ | 6-fluoro-2-chlorphenyl |
| A-52 | CH₃ | 2-chloro-3-methoxyphenyl |
| A-53 | CH₃ | 2-chloro-6-methoxyphenyl |
| A-54 | CH₃ | 2-methyl-6-methoxyphenyl |
| A-55 | CH₃ | 2,3-difluorophenyl |
| A-56 | CH₃ | 2,4-difluorophenyl |
| A-57 | CH₃ | 2,5-difluorophenyl |
| A-58 | CH₃ | 3,4-difluorophenyl |
| A-59 | CH₃ | 3,5-difluorophenyl |
| A-60 | CH₃ | 2,6-difluorophenyl |
| A-61 | CH₃ | 2,4,6-trifluorophenyl |
| A-62 | CH₃ | 3,4,5-trifluorophenyl |
| A-63 | CH₃ | 2,3-dichlorophenyl |
| A-64 | CH₃ | 2,5-dichlorophenyl |
| A-65 | CH₃ | 3,5-dichlorophenyl |
| A-66 | CH₃ | 2,6-dichlorophenyl |
| A-67 | CH₃ | 2,3-dimethylphenyl |
| A-68 | CH₃ | 2,4-dimethylphenyl |
| A-69 | CH₃ | 2,5-dimethylphenyl |
| A-70 | CH₃ | 2,3-dimethoxyphenyl |
| A-71 | CH₃ | 2,4-dimethoxyphenyl |
| A-72 | CH₃ | 2,4-bis(trifluoromethyl)phenyl |
| A-73 | CH₃ | 3,5-bis(trifluoromethyl)phenyl |
| A-74 | CH₃ | 2-methyl-3-methoxyphenyl |
| A-75 | CH₃ | 2-methyl-4-methoxyphenyl |
| A-76 | CH₃ | 3-chloro-4-fluorophenyl |
| A-77 | CH₃ | 2-chloro-4-fluorophenyl |
| A-78 | CH₃ | 4-chloro-2-fluorophenyl |
| A-79 | CH₃ | 4-fluoro-3-methylphenyl |
| A-80 | CH₃ | 2-fluoro-4-methylphenyl |
| A-81 | CH₃ | 4-fluoro-2-methylphenyl |
| A-82 | CH₃ | 2-fluoro-4-methoxyphenyl |
| A-83 | CH₃ | 2-fluoro-4-trifluoromethylphenyl |
| A-84 | CH₃ | 4-chloro-3-methylphenyl |
| A-85 | CH₃ | 2-chloro-4-methylphenyl |
| A-86 | CH₃ | 2-chloro-4-methoxyphenyl |
| A-87 | CH₃ | 2-chloro-4-trifluoromethylphenyl |
| A-88 | CH₃ | 3-fluoro-4-methylphenyl |
| A-89 | CH₃ | 4-fluoro-3-methylphenyl |
| A-90 | CH₃ | 3-fluoro-4-methoxyphenyl |
| A-91 | CH₃ | 3-fluoro-4-ethoxyphenyl |
| A-92 | CH₃ | 3-fluoro-4-trifluoromethylphenyl |
| A-93 | CH₃ | 3-chloro-4-methylphenyl |
| A-94 | CH₃ | 3-chloro-4-methoxyphenyl |
| A-95 | CH₃ | 3-chloro-4-ethoxyphenyl |
| A-96 | CH₃ | 3-chloro-4-trifluoromethylphenyl |
| A-97 | CH₃ | 3-methyl-4-methoxy |
| A-98 | CH₃ | 4-chloro-2,5-difluorophenyl |
| A-99 | CH₃ | 2,6-difluoro-4-trifluoromethylphenyl |
| A-100 | CH₃ | 2,6-dichloro-4-trifluoromethylphenyl |
| A-101 | CH₃ | 4-cyano-2-fluorophenyl |
| A-102 | CH₃ | 4-aminocarbonyl-2-fluorophenyl |
| A-103 | CH₃ | 2-fluoro-4-formylphenyl |
| A-104 | CH₃ | 4-tert-butyl-2-fluorophenyl |
| A-105 | CH₃ | 2-fluoro-4-isopropylphenyl |
| A-106 | CH₃ | 4-ethoxy-2-fluorophenyl |
| A-107 | CH₃ | 4-acetyl-2-fluorophenyl |
| A-108 | CH₃ | 4-methoxycarbonyl-2-fluorophenyl |
| A-109 | CH₃ | 4-ethoxycarbonyl-2-fluorophenyl |
| A-110 | CH₃ | 4-tert-butoxycarbonyl-2-fluorophenyl |
| A-111 | F | phenyl |
| A-112 | F | 2-fluorophenyl |
| A-113 | F | 3-fluorophenyl |
| A-114 | F | 4-fluorophenyl |
| A-115 | F | 2-chlorophenyl |
| A-116 | F | 3-chlorophenyl |
| A-117 | F | 4-chlorophenyl |
| A-118 | F | 2-trifluoromethylphenyl |
| A-119 | F | 3-trifluoromethylphenyl |
| A-120 | F | 4-trifluoromethylphenyl |
| A-121 | F | 2-(methylthio)phenyl |
| A-122 | F | 3-(methylthio)phenyl |
| A-123 | F | 4-(methylthio)phenyl |
| A-124 | F | 2-methoxyphenyl |
| A-125 | F | 3-methoxyphenyl |
| A-126 | F | 4-methoxyphenyl |
| A-127 | F | 3-nitrophenyl |
| A-128 | F | 4-nitrophenyl |
| A-129 | F | 4-cyanophenyl |
| A-130 | F | 4-aminocarbonylphenyl |
| A-131 | F | 4-formylphenyl |
| A-132 | F | 4-tert-butylphenyl |
| A-133 | F | 4-isopropylphenyl |
| A-134 | F | 4-ethoxyphenyl |
| A-135 | F | 4-n-propoxyphenyl |
| A-136 | F | 4-isopropoxyphenyl |
| A-137 | F | 4-n-butoxyphenyl |
| A-138 | F | 4-tert-butoxyphenyl |
| A-139 | F | 4-acetylphenyl |
| A-140 | F | 4-methoxycarbonylphenyl |
| A-141 | F | 4-ethoxycarbonylphenyl |
| A-142 | F | 4-tert-butoxycarbonylphenyl |
| A-143 | F | 4-(methoxyiminomethyl)phenyl |
| A-144 | F | 4-(1-(methoxyimino)ethyl)phenyl |
| A-145 | F | 3-isopropoxyphenyl |
| A-146 | F | 3-ethoxyphenyl |
| A-147 | F | 3-bromophenyl |
| A-148 | F | 4-bromophenyl |
| A-149 | F | 3,4-dimethoxyphenyl |
| A-150 | F | 3-fluoro-4-isopropylphenyl |
| A-151 | F | 3-chloro-4-isopropylphenyl |
| A-152 | F | 2,3,5-trifluorophenyl |
| A-153 | F | 2,4,5-trifluorophenyl |
| A-154 | F | 2,3,4-trifluorophenyl |
| A-155 | F | 2,4,5-trimethylphenyl |
| A-156 | F | 2-fluoro-3-methoxyphenyl |
| A-157 | F | 2-fluoro-6-methoxyphenyl |
| A-158 | F | 5-chloro-2-methylphenyl |
| A-159 | F | 3-chloro-2-methylphenyl |
| A-160 | F | 2-fluoro-5-chlorphenyl |
| A-161 | F | 6-fluoro-2-chlorphenyl |
| A-162 | F | 2-chloro-3-methoxyphenyl |
| A-163 | F | 2-chloro-6-methoxyphenyl |
| A-164 | F | 2-methyl-6-methoxyphenyl |
| A-165 | F | 2,3-difluorophenyl |
| A-166 | F | 2,4-difluorophenyl |
| A-167 | F | 2,5-difluorophenyl |
| A-168 | F | 3,4-difluorophenyl |
| A-169 | F | 3,5-difluorophenyl |
| A-170 | F | 2,6-difluorophenyl |
| A-171 | F | 2,4,6-trifluorophenyl |
| A-172 | F | 3,4,5-trifluorophenyl |
| A-173 | F | 2,3-dichlorophenyl |
| A-174 | F | 2,5-dichlorophenyl |
| A-175 | F | 3,5-dichlorophenyl |
| A-176 | F | 2,6-dichlorophenyl |
| A-177 | F | 2,3-dimethylphenyl |
| A-178 | F | 2,4-dimethylphenyl |
| A-179 | F | 2,5-dimethylphenyl |
| A-180 | F | 2,3-dimethoxyphenyl |
| A-181 | F | 2,4-dimethoxyphenyl |
| A-182 | F | 2,4-bis(trifluoromethyl)phenyl |
| A-183 | F | 3,5-bis(trifluoromethyl)phenyl |
| A-184 | F | 2-methyl-3-methoxyphenyl |
| A-185 | F | 2-methyl-4-methoxyphenyl |
| A-186 | F | 3-chloro-4-fluorophenyl |
| A-187 | F | 2-chloro-4-fluorophenyl |
| A-188 | F | 4-chloro-2-fluorophenyl |
| A-189 | F | 4-fluoro-3-methylphenyl |
| A-190 | F | 2-fluoro-4-methylphenyl |
| A-191 | F | 4-fluoro-2-methylphenyl |
| A-192 | F | 2-fluoro-4-methoxyphenyl |
| A-193 | F | 2-fluoro-4-trifluoromethylphenyl |
| A-194 | F | 4-chloro-3-methylphenyl |
| A-195 | F | 2-chloro-4-methylphenyl |
| A-196 | F | 2-chloro-4-methoxyphenyl |
| A-197 | F | 2-chloro-4-trifluoromethylphenyl |
| A-198 | F | 3-fluoro-4-methylphenyl |
| A-199 | F | 4-fluoro-3-methylphenyl |
| A-200 | F | 3-fluoro-4-methoxyphenyl |
| A-201 | F | 3-fluoro-4-ethoxyphenyl |
| A-202 | F | 3-fluoro-4-trifluoromethylphenyl |
| A-203 | F | 3-chloro-4-methylphenyl |
| A-204 | F | 3-chloro-4-methoxyphenyl |
| A-205 | F | 3-chloro-4-ethoxyphenyl |
| A-206 | F | 3-chloro-4-trifluoromethylphenyl |
| A-207 | F | 3-methyl-4-methoxy |
| A-208 | F | 4-chloro-2,5-difluorophenyl |
| A-209 | F | 2,6-difluoro-4-trifluoromethylphenyl |
| A-210 | F | 2,6-dichloro-4-trifluoromethylphenyl |
| A-211 | F | 4-cyano-2-fluorophenyl |
| A-212 | F | 4-aminocarbonyl-2-fluorophenyl |
| A-213 | F | 2-fluoro-4-formylphenyl |
| A-214 | F | 4-tert-butyl-2-fluorophenyl |
| A-215 | F | 2-fluoro-4-isopropylphenyl |
| A-216 | F | 4-ethoxy-2-fluorophenyl |
| A-217 | F | 4-acetyl-2-fluorophenyl |
| A-218 | F | 4-methoxycarbonyl-2-fluorophenyl |
| A-219 | F | 4-ethoxycarbonyl-2-fluorophenyl |
| A-220 | F | 4-tert-butoxycarbonyl-2-fluorophenyl |
| A-221 | OCH₃ | phenyl |
| A-222 | OCH₃ | 2-fluorophenyl |
| A-223 | OCH₃ | 3-fluorophenyl |
| A-224 | OCH₃ | 4-fluorophenyl |
| A-225 | OCH₃ | 2-chlorophenyl |
| A-226 | OCH₃ | 3-chlorophenyl |
| A-227 | OCH₃ | 4-chlorophenyl |
| A-228 | OCH₃ | 2-trifluoromethylphenyl |
| A-229 | OCH₃ | 3-trifluoromethylphenyl |
| A-230 | OCH₃ | 4-trifluoromethylphenyl |
| A-231 | OCH₃ | 2-(methylthio)phenyl |
| A-232 | OCH₃ | 3-(methylthio)phenyl |
| A-233 | OCH₃ | 4-(methylthio)phenyl |
| A-234 | OCH₃ | 2-methoxyphenyl |
| A-235 | OCH₃ | 3-methoxyphenyl |
| A-236 | OCH₃ | 4-methoxyphenyl |
| A-237 | OCH₃ | 3-nitrophenyl |
| A-238 | OCH₃ | 4-nitrophenyl |
| A-239 | OCH₃ | 4-cyanophenyl |
| A-240 | OCH₃ | 4-aminocarbonylphenyl |
| A-241 | OCH₃ | 4-formylphenyl |
| A-242 | OCH₃ | 4-tert-butylphenyl |
| A-243 | OCH₃ | 4-isopropylphenyl |
| A-244 | OCH₃ | 4-ethoxyphenyl |
| A-245 | OCH₃ | 4-n-propoxyphenyl |
| A-246 | OCH₃ | 4-isopropoxyphenyl |
| A-247 | OCH₃ | 4-n-butoxyphenyl |
| A-248 | OCH₃ | 4-tert-butoxyphenyl |
| A-249 | OCH₃ | 4-acetylphenyl |
| A-250 | OCH₃ | 4-methoxycarbonylphenyl |
| A-251 | OCH₃ | 4-ethoxycarbonylphenyl |
| A-252 | OCH₃ | 4-tert-butoxycarbonylphenyl |
| A-253 | OCH₃ | 4-(methoxyiminomethyl)phenyl |
| A-254 | OCH₃ | 4-(1-(methoxyimino)ethyl)phenyl |
| A-255 | OCH₃ | 3-isopropoxyphenyl |
| A-256 | OCH₃ | 3-ethoxyphenyl |
| A-257 | OCH₃ | 3-bromophenyl |
| A-258 | OCH₃ | 4-bromophenyl |
| A-259 | OCH₃ | 3,4-dimethoxyphenyl |
| A-260 | OCH₃ | 3-fluoro-4-isopropylphenyl |
| A-261 | OCH₃ | 3-chloro-4-isopropylphenyl |
| A-262 | OCH₃ | 2,3,5-trifluorophenyl |
| A-263 | OCH₃ | 2,4,5-trifluorophenyl |
| A-264 | OCH₃ | 2,3,4-trifluorophenyl |
| A-265 | OCH₃ | 2,4,5-trimethylphenyl |
| A-266 | OCH₃ | 2-fluoro-3-methoxyphenyl |
| A-267 | OCH₃ | 2-fluoro-6-methoxyphenyl |
| A-268 | OCH₃ | 5-chloro-2-methylphenyl |
| A-269 | OCH₃ | 3-chloro-2-methylphenyl |
| A-270 | OCH₃ | 2-fluoro-5-chlorophenyl |
| A-271 | OCH₃ | 6-fluoro-2-chlorophenyl |
| A-272 | OCH₃ | 2-chloro-3-methoxyphenyl |
| A-273 | OCH₃ | 2-chloro-6-methoxyphenyl |
| A-274 | OCH₃ | 2-methyl-6-methoxyphenyl |
| A-275 | OCH₃ | 2,3-difluorophenyl |
| A-276 | OCH₃ | 2,4-difluorophenyl |
| A-277 | OCH₃ | 2,5-difluorophenyl |
| A-278 | OCH₃ | 3,4-difluorophenyl |
| A-279 | OCH₃ | 3,5-difluorophenyl |
| A-280 | OCH₃ | 2,6-difluorophenyl |
| A-281 | OCH₃ | 2,4,6-trifluorophenyl |
| A-282 | OCH₃ | 3,4,5-trifluorophenyl |
| A-283 | OCH₃ | 2,3-dichlorophenyl |
| A-284 | OCH₃ | 2,5-dichlorophenyl |
| A-285 | OCH₃ | 3,5-dichlorophenyl |
| A-286 | OCH₃ | 2,6-dichlorophenyl |
| A-287 | OCH₃ | 2,3-dimethylphenyl |
| A-288 | OCH₃ | 2,4-dimethylphenyl |
| A-289 | OCH₃ | 2,5-dimethylphenyl |
| A-290 | OCH₃ | 2,3-dimethoxyphenyl |
| A-291 | OCH₃ | 2,4-dimethoxyphenyl |
| A-292 | OCH₃ | 2,4-bis(trifluoromethyl)phenyl |
| A-293 | OCH₃ | 3,5-bis(trifluoromethyl)phenyl |
| A-294 | OCH₃ | 2-methyl-3-methoxyphenyl |
| A-295 | OCH₃ | 2-methyl-4-methoxyphenyl |
| A-296 | OCH₃ | 3-chloro-4-fluorophenyl |
| A-297 | OCH₃ | 2-chloro-4-fluorophenyl |
| A-298 | OCH₃ | 4-chloro-2-fluorophenyl |
| A-299 | OCH₃ | 4-fluoro-3-methylphenyl |
| A-300 | OCH₃ | 2-fluoro-4-methylphenyl |
| A-301 | OCH₃ | 4-fluoro-2-methylphenyl |
| A-302 | OCH₃ | 2-fluoro-4-methoxyphenyl |
| A-303 | OCH₃ | 2-fluoro-4-trifluoromethylphenyl |
| A-304 | OCH₃ | 4-chloro-3-methylphenyl |
| A-305 | OCH₃ | 2-chloro-4-methylphenyl |
| A-306 | OCH₃ | 2-chloro-4-methoxyphenyl |
| A-307 | OCH₃ | 2-chloro-4-trifluoromethylphenyl |
| A-308 | OCH₃ | 3-fluoro-4-methylphenyl |
| A-309 | OCH₃ | 4-fluoro-3-methylphenyl |
| A-310 | OCH₃ | 3-fluoro-4-methoxyphenyl |
| A-311 | OCH₃ | 3-fluoro-4-ethoxyphenyl |
| A-312 | OCH₃ | 3-fluoro-4-trifluoromethylphenyl |
| A-313 | OCH₃ | 3-chloro-4-methylphenyl |
| A-314 | OCH₃ | 3-chloro-4-methoxyphenyl |
| A-315 | OCH₃ | 3-chloro-4-ethoxyphenyl |
| A-316 | OCH₃ | 3-chloro-4-trifluormethylphenyl |
| A-317 | OCH₃ | 3-methyl-4-methoxy |
| A-318 | OCH₃ | 4-chloro-2,5-difluorophenyl |
| A-319 | OCH₃ | 2,6-difluoro-4-trifluoromethylphenyl |
| A-320 | OCH₃ | 2,6-dichloro-4-trifluoromethylphenyl |
| A-321 | OCH₃ | 4-cyano-2-fluorophenyl |
| A-322 | OCH₃ | 4-aminocarbonyl-2-fluorophenyl |
| A-323 | OCH₃ | 2-fluoro-4-formylphenyl |
| A-324 | OCH₃ | 4-tert-butyl-2-fluorophenyl |
| A-325 | OCH₃ | 2-fluoro-4-isopropylphenyl |
| A-326 | OCH₃ | 4-ethoxy-2-fluorophenyl |
| A-327 | OCH₃ | 4-acetyl-2-fluorophenyl |
| A-328 | OCH₃ | 4-methoxycarbonyl-2-fluorophenyl |
| A-329 | OCH₃ | 4-ethoxycarbonyl-2-fluorophenyl |
| A-330 | OCH₃ | 4-tert-butoxycarbonyl-2-fluorophenyl |
| A-331 | CH₃ | 5-methylthiophen-2-yl |
| A-332 | CH₃ | 4-methylthiophen-2-yl |
| A-333 | CH₃ | 5-chlorothiophen-2-yl |
| A-334 | CH₃ | 3-cyanothiophen-2-yl |
| A-335 | CH₃ | 4-bromothiophen-2-yl |
| A-336 | CH₃ | 3,5-dichlorothiophen-2-yl |
| A-337 | CH₃ | 3,4,5-trichlorothiophen-2-yl |
| A-338 | CH₃ | 5-bromothiophen-2-yl |
| A-339 | CH₃ | 3-thienyl |
| A-340 | CH₃ | 2-methylthiophen-3-yl |
| A-341 | CH₃ | 2,5-dichlorothiophen-3-yl |
| A-342 | CH₃ | 2-furyl |
| A-343 | CH₃ | 5-methylfuran-2-yl |
| A-344 | CH₃ | 5-chlorofuran-2-yl |
| A-345 | CH₃ | 4-methylfuran-2-yl |
| A-346 | CH₃ | 3-cyanofuran-2-yl |
| A-347 | CH₃ | 5-acetylfuran-2-yl |
| A-348 | CH₃ | 3,5-dichlorofuran-2-yl |
| A-349 | CH₃ | 5-bromofuran-2-yl |
| A-350 | CH₃ | 3-furyl |
| A-351 | CH₃ | 2-methylfuran-3-yl |
| A-352 | CH₃ | 2,5-dimethylfuran-3-yl |
| A-353 | CH₃ | 2-pyridyl |
| A-354 | CH₃ | 3-fluoropyridin-2-yl |
| A-355 | CH₃ | 3-chloropyridin-2-yl |
| A-356 | CH₃ | 3-bromo-2-pyridin-2-yl |
| A-357 | CH₃ | 3-trifluoromethylpyridin-2-yl |
| A-358 | CH₃ | 3-methylpyridin-2-yl |
| A-359 | CH₃ | 3-ethylpyridin-2-yl |
| A-360 | CH₃ | 3,5-difluoropyridin-2-yl |
| A-361 | CH₃ | 3,5-dichloropyridin-2-yl |
| A-362 | CH₃ | 3,5-dibromopyridin-2-yl |
| A-363 | CH₃ | 3,5-dimethylpyridin-2-yl |
| A-364 | CH₃ | 3-fluoro-5-trifluoromethylpyridin-2-yl |
| A-365 | CH₃ | 3-chloro-5-fluoropyridin-2-yl |
| A-366 | CH₃ | 3-chloro-5-methylpyridin-2-yl |
| A-367 | CH₃ | 3-fluoro-5-chloropyridin-2-yl |
| A-368 | CH₃ | 3-fluoro-5-methylpyridin-2-yl |
| A-369 | CH₃ | 3-methyl-5-fluoropyridin-2-yl |
| A-370 | CH₃ | 3-methyl-5-chloropyridin-2-yl |
| A-371 | CH₃ | 5-nitropyridin-2-yl |
| A-372 | CH₃ | 5-cyanopyridin-2-yl |
| A-373 | CH₃ | 5-methoxycarbonylpyridin-2-yl |
| A-374 | CH₃ | 5-trifluoromethylpyridin-2-yl |
| A-375 | CH₃ | 5-methylpyridin-2-yl |
| A-376 | CH₃ | 4-methylpyridin-2-yl |
| A-377 | CH₃ | 6-methylpyridin-2-yl |
| A-378 | CH₃ | 3-pyridyl |
| A-379 | CH₃ | 2-chloropyridin-3-yl |
| A-380 | CH₃ | 2-bromopyridin-3-yl |
| A-381 | CH₃ | 2-methylpyridin-3-yl |
| A-382 | CH₃ | 2,4-dichloropyridin-3-yl |
| A-383 | CH₃ | 2,4-dibromopyridin-3-yl |
| A-384 | CH₃ | 2,4-difluoropyridin-3-yl |
| A-385 | CH₃ | 2-fluoro-4-chloropyridin-3-yl |
| A-386 | CH₃ | 2-chloro-4-fluoropyrdin-3-yl |
| A-387 | CH₃ | 2-chloro-4-methylpyridin-3-yl |
| A-388 | CH₃ | 2-methyl-4-fluoropyridin-3-yl |
| A-389 | CH₃ | 2-methyl-4-chloropyridin-3-yl |
| A-390 | CH₃ | 2,4-dimethylpyridin-3-yl |
| A-391 | CH₃ | 2,4,6-trichloropyridin-3-yl |
| A-392 | CH₃ | 2,4,6-tribromopyridin-3-yl |
| A-393 | CH₃ | 2,4,6-trimethylpyridin-3-yl |
| A-394 | CH₃ | 2,4-dichloro-6-methylpyridin-3-yl |
| A-395 | CH₃ | 4-pyridyl |
| A-396 | CH₃ | 3-chloropyridin-4-yl |
| A-397 | CH₃ | 3-bromopyridin-4-yl |
| A-398 | CH₃ | 3-methylpyridin-4-yl |
| A-399 | CH₃ | 3,5-dichloropyridin-4-yl |
| A-400 | CH₃ | 3,5-dibromopyridin-4-yl |
| A-401 | CH₃ | 3,5-dimethylpyridin-4-yl |
| A-402 | CH₃ | 4-pyrimidinyl |
| A-403 | CH₃ | 5-chloropyrimidin-4-yl |
| A-404 | CH₃ | 5-fluoropyrimidin-4-yl |
| A-405 | CH₃ | 5-fluoro-6-chloropyrimidin-4-yl |
| A-406 | CH₃ | 2-methyl-6-trifluoromethylpyrimidin-4-yl |
| A-407 | CH₃ | 2,5-dimethyl-6-trifluoromethylpyrimidin-4-yl |
| A-408 | CH₃ | 5-methyl-6-trifluoromethylpyrimidin-4-yl |
| A-409 | CH₃ | 6-trifluoromethylpyrimidin-4-yl |
| A-410 | CH₃ | 2-methyl-5-fluoropyrimidin-4-yl |
| A-411 | CH₃ | 2-methyl-5-chloropyrimidin-4-yl |
| A-412 | CH₃ | 5-chloro-6-methylpyrimidin-4-yl |
| A-413 | CH₃ | 5-chloro-6-ethylpyrimidin-4-yl |
| A-414 | CH₃ | 5-chloro-6-isopropylpyrimidin-4-yl |
| A-415 | CH₃ | 5-bromo-6-methylpyrimidin-4-yl |
| A-416 | CH₃ | 5-fluoro-6-methylpyrimidin-4-yl |
| A-417 | CH₃ | 5-fluoro-6-fluoromethylpyrimidin-4-yl |
| A-418 | CH₃ | 2,6-dimethyl-5-chloropyrimidin-4-yl |
| A-419 | CH₃ | 5,6-dimethylpyrimidin-4-yl |
| A-420 | CH₃ | 2,5-dimethylpyrimidin-4-yl |
| A-421 | CH₃ | 2,5,6-trimethylpyrimidin-4-yl |
| A-422 | CH₃ | 5-methyl-6-methoxypyrimidin-4-yl |
| A-423 | CH₃ | 5-pyrimidinyl |
| A-424 | CH₃ | 4-methylpyrimidin-5-yl |
| A-425 | CH₃ | 4,6-dimethylpyrimidin-5-yl |
| A-426 | CH₃ | 2,4,6-trimethylpyrimidin-5-yl |
| A-427 | CH₃ | 4-trifluoromethyl-6-methylpyrimidin-5-yl |
| A-428 | CH₃ | 2-pyrimidinyl |
| A-429 | CH₃ | 4,6-dimethylpyrimidin-2-yl |
| A-430 | CH₃ | 4,5,6-trimethylpyrimidin-2-yl |
| A-431 | CH₃ | 4,6-ditrifluoromethylpyrimidin-2-yl |
| A-432 | CH₃ | 4,6-dimethyl-5-chloropyrimidin-2-yl |
| A-433 | OCH₃ | 2-thienyl |
| A-434 | OCH₃ | 5-methylthiophen-2-yl |
| A-435 | OCH₃ | 4-methylthiophen-2-yl |
| A-436 | OCH₃ | 5-chlorothiophen-2-yl |
| A-437 | OCH₃ | 3-cyanothiophen-2-yl |
| A-438 | OCH₃ | 4-bromothiophen-2-yl |
| A-439 | OCH₃ | 3,5-dichlorothiophen-2-yl |
| A-440 | OCH₃ | 3,4,5-trichlorothiophen-2-yl |
| A-441 | OCH₃ | 5-bromothiophen-2-yl |
| A-442 | OCH₃ | 3-thienyl |
| A-443 | OCH₃ | 2-methylthiophen-3-yl |
| A-444 | OCH₃ | 2,5-dichlorothiophen-3-yl |
| A-445 | OCH₃ | 2-furyl |
| A-446 | OCH₃ | 5-methylfuran-2-yl |
| A-447 | OCH₃ | 5-chlorofuran-2-yl |
| A-448 | OCH₃ | 4-methylfuran-2-yl |
| A-449 | OCH₃ | 3-cyanofuran-2-yl |
| A-450 | OCH₃ | 5-acetylfuran-2-yl |
| A-451 | OCH₃ | 3,5-dichlorofuran-2-yl |
| A-452 | OCH₃ | 5-bromofuran-2-yl |
| A-453 | OCH₃ | 3-furyl |
| A-454 | OCH₃ | 2-methylfuran-3-yl |
| A-455 | OCH₃ | 2,5-dimethylfuran-3-yl |
| A-456 | OCH₃ | 2-pyridyl |
| A-457 | OCH₃ | 3-fluoropyridin-2-yl |
| A-458 | OCH₃ | 3-chloropyridin-2-yl |
| A-459 | OCH₃ | 3-bromo-2-pyridin-2-yl |
| A-460 | OCH₃ | 3-trifluoromethylpyridin-2-yl |
| A-461 | OCH₃ | 3-methylpyridin-2-yl |
| A-462 | OCH₃ | 3-ethylpyridin-2-yl |
| A-463 | OCH₃ | 3,5-difluoropyridin-2-yl |
| A-464 | OCH₃ | 3,5-dichloropyridin-2-yl |
| A-465 | OCH₃ | 3,5-dibromopyridin-2-yl |
| A-466 | OCH₃ | 3,5-dimethylpyridin-2-yl |
| A-467 | OCH₃ | 3-fluoro-5-trifluoromethylpyridin-2-yl |
| A-468 | OCH₃ | 3-chloro-5-fluoropyridin-2-yl |
| A-469 | OCH₃ | 3-chloro-5-methylpyridin-2-yl |
| A-470 | OCH₃ | 3-fluoro-5-chloropyridin-2-yl |
| A-471 | OCH₃ | 3-fluoro-5-methylpyridin-2-yl |
| A-472 | OCH₃ | 3-methyl-5-fluoropyridin-2-yl |
| A-473 | OCH₃ | 3-methyl-5-chloropyridin-2-yl |
| A-474 | OCH₃ | 5-nitropyridin-2-yl |
| A-475 | OCH₃ | 5-cyanopyridin-2-yl |
| A-476 | OCH₃ | 5-methoxycarbonylpyridin-2-yl |
| A-477 | OCH₃ | 5-trifluoromethylpyridin-2-yl |
| A-478 | OCH₃ | 5-methylpyridin-2-yl |
| A-479 | OCH₃ | 4-methylpyridin-2-yl |
| A-480 | OCH₃ | 6-methylpyridin-2-yl |
| A-481 | OCH₃ | 3-pyridyl |
| A-482 | OCH₃ | 2-chloropyridin-3-yl |
| A-483 | OCH₃ | 2-bromopyridin-3-yl |
| A-484 | OCH₃ | 2-methylpyridin-3-yl |
| A-485 | OCH₃ | 2,4-dichloropyridin-3-yl |
| A-486 | OCH₃ | 2,4-dibromopyridin-3-yl |
| A-487 | OCH₃ | 2,4-difluoropyridin-3-yl |
| A-488 | OCH₃ | 2-fluoro-4-chloropyridin-3-yl |
| A-489 | OCH₃ | 2-chloro-4-fluoropyrdin-3-yl |
| A-490 | OCH₃ | 2-chloro-4-methylpyridin-3-yl |
| A-491 | OCH₃ | 2-methyl-4-fluoropyridin-3-yl |
| A-492 | OCH₃ | 2-methyl-4-chloropyridin-3-yl |
| A-493 | OCH₃ | 2,4-dimethylpyridin-3-yl |
| A-494 | OCH₃ | 2,4,6-trichloropyridin-3-yl |
| A-495 | OCH₃ | 2,4,6-tribromopyridin-3-yl |
| A-496 | OCH₃ | 2,4,6-trimethylpyridin-3-yl |
| A-497 | OCH₃ | 2,4-dichloro-6-methylpyridin-3-yl |
| A-498 | OCH₃ | 4-pyridyl |
| A-499 | OCH₃ | 3-chloropyridin-4-yl |
| A-500 | OCH₃ | 3-bromopyridin-4-yl |
| A-501 | OCH₃ | 3-methylpyridin-4-yl |
| A-502 | OCH₃ | 3,5-dichloropyridin-4-yl |
| A-503 | OCH₃ | 3,5-dibromopyridin-4-yl |
| A-504 | OCH₃ | 3,5-dimethylpyridin-4-yl |
| A-505 | OCH₃ | 4-pyrimidinyl |
| A-506 | OCH₃ | 5-chloropyrimidin-4-yl |
| A-507 | OCH₃ | 5-fluoropyrimidin-4-yl |
| A-508 | OCH₃ | 5-fluoro-6-chloropyrimidin-4-yl |
| A-509 | OCH₃ | 2-methyl-6-trifluoromethylpyrimidin-4-yl |
| A-510 | OCH₃ | 2,5-dimethyl-6-trifluoromethylpyrimidin-4-yl |
| A-511 | OCH₃ | 5-methyl-6-trifluoromethylpyrimidin-4-yl |
| A-512 | OCH₃ | 6-trifluoromethylpyrimidin-4-yl |
| A-513 | OCH₃ | 2-methyl-5-fluoropyrimidin-4-yl |
| A-514 | OCH₃ | 2-methyl-5-chloropyrimidin-4-yl |
| A-515 | OCH₃ | 5-chloro-6-methylpyrimidin-4-yl |
| A-516 | OCH₃ | 5-chloro-6-ethylpyrimidin-4-yl |
| A-517 | OCH₃ | 5-chloro-6-isopropylpyrimidin-4-yl |
| A-518 | OCH₃ | 5-bromo-6-methylpyrimidin-4-yl |
| A-519 | OCH₃ | 5-fluoro-6-methylpyrimidin-4-yl |
| A-520 | OCH₃ | 5-fluoro-6-fluoromethylpyrimidin-4-yl |
| A-521 | OCH₃ | 2,6-dimethyl-5-chloropyrimidin-4-yl |
| A-522 | OCH₃ | 5,6-dimethylpyrimidin-4-yl |
| A-523 | OCH₃ | 2,5-dimethylpyrimidin-4-yl |
| A-524 | OCH₃ | 2,5,6-trimethylpyrimidin-4-yl |
| A-525 | OCH₃ | 5-methyl-6-methoxypyrimidin-4-yl |
| A-526 | OCH₃ | 5-pyrimidinyl |
| A-527 | OCH₃ | 4-methylpyrimidin-5-yl |
| A-528 | OCH₃ | 4,6-dimethylpyrimidin-5-yl |
| A-529 | OCH₃ | 2,4,6-trimethylpyrimidin-5-yl |
| A-530 | OCH₃ | 4-trifluoromethyl-6-methylpyrimidin-5-yl |
| A-531 | OCH₃ | 2-pyrimidinyl |
| A-532 | OCH₃ | 4,6-dimethylpyrimidin-2-yl |
| A-533 | OCH₃ | 4,5,6-trimethylpyrimidin-2-yl |
| A-534 | OCH₃ | 4,6-ditrifluoromethylpyrimidin-2-yl |
| A-535 | OCH₃ | 4,6-dimethyl-5-chloropyrimidin-2-yl |
| A-536 | F | 2-thienyl |
| A-537 | F | 5-methylthiophen-2-yl |
| A-538 | F | 4-methylthiophen-2-yl |
| A-539 | F | 5-chlorothiophen-2-yl |
| A-540 | F | 3-cyanothiophen-2-yl |
| A-541 | F | 4-bromothiophen-2-yl |
| A-542 | F | 3,5-dichlorothiophen-2-yl |
| A-543 | F | 3,4,5-trichlorothiophen-2-yl |
| A-544 | F | 5-bromothiophen-2-yl |
| A-545 | F | 3-thienyl |
| A-546 | F | 2-methylthiophen-3-yl |
| A-547 | F | 2,5-dichlorothiophen-3-yl |
| A-548 | F | 2-furyl |
| A-549 | F | 5-methylfuran-2-yl |
| A-550 | F | 5-chlorofuran-2-yl |
| A-551 | F | 4-methylfuran-2-yl |
| A-552 | F | 3-cyanofuran-2-yl |
| A-553 | F | 5-acetylfuran-2-yl |
| A-554 | F | 3,5-dichlorofuran-2-yl |
| A-555 | F | 5-bromofuran-2-yl |
| A-556 | F | 3-furyl |
| A-557 | F | 2-methylfuran-3-yl |
| A-558 | F | 2,5-dimethylfuran-3-yl |
| A-559 | F | 2-pyridyl |
| A-560 | F | 3-fluoropyridin-2-yl |
| A-561 | F | 3-chloropyridin-2-yl |
| A-562 | F | 3-bromo-2-pyridin-2-yl |
| A-563 | F | 3-trifluoromethylpyridin-2-yl |
| A-564 | F | 3-methylpyridin-2-yl |
| A-565 | F | 3-ethylpyridin-2-yl |
| A-566 | F | 3,5-difluoropyridin-2-yl |
| A-567 | F | 3,5-dichloropyridin-2-yl |
| A-568 | F | 3,5-dibromopyridin-2-yl |
| A-569 | F | 3,5-dimethylpyridin-2-yl |
| A-570 | F | 3-fluoro-5-trifluoromethylpyridin-2-yl |
| A-571 | F | 3-chloro-5-fluoropyridin-2-yl |
| A-572 | F | 3-chloro-5-methylpyridin-2-yl |
| A-573 | F | 3-fluoro-5-chloropyridin-2-yl |
| A-574 | F | 3-fluoro-5-methylpyridin-2-yl |
| A-575 | F | 3-methyl-5-fluoropyridin-2-yl |
| A-576 | F | 3-methyl-5-chloropyridin-2-yl |
| A-577 | F | 5-nitropyridin-2-yl |
| A-578 | F | 5-cyanopyridin-2-yl |
| A-579 | F | 5-methoxycarbonylpyridin-2-yl |
| A-580 | F | 5-trifluoromethylpyridin-2-yl |
| A-581 | F | 5-methylpyridin-2-yl |
| A-582 | F | 4-methylpyridin-2-yl |
| A-583 | F | 6-methylpyridin-2-yl |
| A-584 | F | 3-pyridyl |
| A-585 | F | 2-chloropyridin-3-yl |
| A-586 | F | 2-bromopyridin-3-yl |
| A-587 | F | 2-methylpyridin-3-yl |
| A-588 | F | 2,4-dichloropyridin-3-yl |
| A-589 | F | 2,4-dibromopyridin-3-yl |
| A-590 | F | 2,4-difluoropyridin-3-yl |
| A-591 | F | 2-fluoro-4-chloropyridin-3-yl |
| A-592 | F | 2-chloro-4-fluoropyrdin-3-yl |
| A-593 | F | 2-chloro-4-methylpyridin-3-yl |
| A-594 | F | 2-methyl-4-fluoropyridin-3-yl |
| A-595 | F | 2-methyl-4-chloropyridin-3-yl |
| A-596 | F | 2,4-dimethylpyridin-3-yl |
| A-597 | F | 2,4,6-trichloropyridin-3-yl |
| A-598 | F | 2,4,6-tribromopyridin-3-yl |
| A-599 | F | 2,4,6-trimethylpyridin-3-yl |
| A-600 | F | 2,4-dichloro-6-methylpyridin-3-yl |
| A-601 | F | 4-pyridyl |
| A-602 | F | 3-chloropyridin-4-yl |
| A-603 | F | 3-bromopyridin-4-yl |
| A-604 | F | 3-methylpyridin-4-yl |
| A-605 | F | 3,5-dichloropyridin-4-yl |
| A-606 | F | 3,5-dibromopyridin-4-yl |
| A-607 | F | 3,5-dimethylpyridin-4-yl |
| A-608 | F | 4-pyrimidinyl |
| A-609 | F | 5-chloropyrimidin-4-yl |
| A-610 | F | 5-fluoropyrimidin-4-yl |
| A-611 | F | 5-fluoro-6-chloropyrimidin-4-yl |
| A-612 | F | 2-methyl-6-trifluoromethylpyrimidin-4-yl |
| A-613 | F | 2,5-dimethyl-6-trifluoromethylpyrimidin-4-yl |
| A-614 | F | 5-methyl-6-trifluoromethylpyrimidin-4-yl |
| A-615 | F | 6-trifluoromethylpyrimidin-4-yl |
| A-616 | F | 2-methyl-5-fluoropyrimidin-4-yl |
| A-617 | F | 2-methyl-5-chloropyrimidin-4-yl |
| A-618 | F | 5-chloro-6-methylpyrimidin-4-yl |
| A-619 | F | 5-chloro-6-ethylpyrimidin-4-yl |
| A-620 | F | 5-chloro-6-isopropylpyrimidin-4-yl |
| A-621 | F | 5-bromo-6-methylpyrimidin-4-yl |
| A-622 | F | 5-fluoro-6-methylpyrimidin-4-yl |
| A-623 | F | 5-fluoro-6-fluoromethylpyrimidin-4-yl |
| A-624 | F | 2,6-dimethyl-5-chloropyrimidin-4-yl |
| A-625 | F | 5,6-dimethylpyrimidin-4-yl |
| A-626 | F | 2,5-dimethylpyrimidin-4-yl |
| A-627 | F | 2,5,6-trimethylpyrimidin-4-yl |
| A-628 | F | 5-methyl-6-methoxypyrimidin-4-yl |
| A-629 | F | 5-pyrimidinyl |
| A-630 | F | 4-methylpyrimidin-5-yl |
| A-631 | F | 4,6-dimethylpyrimidin-5-yl |
| A-632 | F | 2,4,6-trimethylpyrimidin-5-yl |
| A-633 | F | 4-trifluoromethyl-6-methylpyrimidin-5-yl |
| A-634 | F | 2-pyrimidinyl |
| A-635 | F | 4,6-dimethylpyrimidin-2-yl |
| A-636 | F | 4,5,6-trimethylpyrimidin-2-yl |
| A-637 | F | 4,6-ditrifluoromethylpyrimidin-2-yl |
| A-638 | F | 4,6-dimethyl-5-chloropyrimidin-2-yl |

The compounds according to the invention of the general formula I can be prepared analogously to the prior art cited at the outset using standard methods of organic synthesis.

Compounds of the formula I can be prepared, for example, using the process shown in Scheme 1:

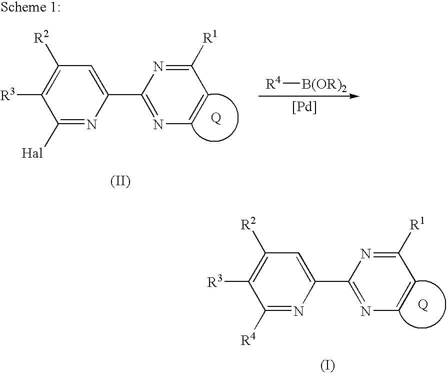

In Scheme 1, Q, R¹, R², R³ and R⁴ are as defined above. R is H or C₁-C₄-alkyl or, together with further molecules R⁴—B(OR)₂, forms a phenylboronic anhydride. Hal is bromine or iodine.

According to Scheme 1, the 2-(6-halopyridin-2-yl)pyrimidine of the formula II is reacted with a (het)arylboronic acid derivative of the general formula R⁴—B(OR)₂ under the conditions of a Suzuki coupling, i.e. in the presence of a palladium catalyst under reaction conditions known per se as disclosed, for example, in Acc. Chem. Res. 15, pp. 178-184 (1982), Chem. Rev. 95, pp. 2457-2483 (1995), and the literature cited therein, and also in J. Org. Chem. 68, p. 9412 (2003). Suitable catalysts are in particular tetrakis(triphenylphosphine)palladium(0), bis(triphenylphosphine)palladium(II) chloride, bis(acetonitrile)palladium(II) chloride, the [1,1′-bis(diphenylphosphino)ferrocene]-palladium(II) chloride/dichloromethane complex, bis[1,2-bis(diphenylphosphine)-ethane]palladium(0) and [1,4-bis(diphenylphosphine)butane]palladium(II) chloride. The amount of catalyst is usually form 0.1 to 10 mol %, based on the compound II. The molar ratio of compound II to the (het)arylboronic acid derivative is typically in the range from 1:2 to 2:1.

Instead of the arylboronic acid derivative, it is also possible to employ organometallic compounds Met-R⁴ in which R⁴ is as defined above and Met is a radical MgX, SnR₃ or ZnX (X=chlorine, bromine or iodine, R=alkyl). In this case, the reaction of II with the compound Met-R⁴ is carried out, for example, in the sense of a Stille coupling or Kumada coupling.

Some of the 3-(6-halopyridin-2-yl)pyrimidines of the formula II are known, for example from WO 2006/010570, or they can be prepared for their part by the methods shown in the schemes below from the corresponding amidine compounds of the formula Ill.

The preparation von compounds II in which Q forms a saturated carbocycle Q-1 according to the above definition can be achieved, for example, according to the synthesis shown in Scheme 2.

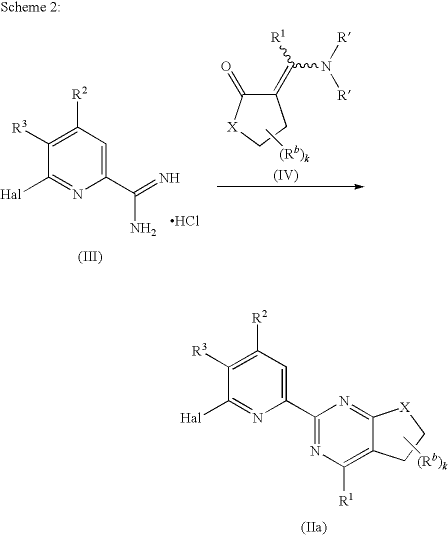

In Scheme 2, R¹, R², R³, Hal, R^{b}, X and k are as defined above. R¹ is in particular hydrogen. R¹ is C₁-C₄-alkyl and in particular methyl.

According to Scheme 2, the pyridin-2-ylamidinium hydrochloride of the formula III is reacted with a dialkylaminomethylenecycloalkanone of the formula IV (enaminoketone IV) in the presence of a base, preferably an alkali metal alkoxide, such as sodium methoxide or sodium ethoxide. The reaction can be carried out analogously to known processes for reacting amidinium hydrochlorides with enaminoketones as described, for example, in J. Heterocycl. Chem. 20, pp. 649-653 (1983). Instead of the enaminoketones IV, it is also possible to use β-oxoacetals of the formula IVa.

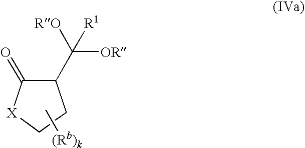

In formula IVa, R″ is C₁-C₄-alkyl and in particular methyl or ethyl. R¹ is in particular hydrogen. The reaction of III with IVa can be carried out analogously to method (a) described in EP-A 259139, with is incorporated herein by way of reference.

Dialkylaminomethylenecycloalkanones of the formula IV are known or can be prepared analogously to known methods [see, for example, WO 2001/087845, Tetrahedron 50(7), pp. 2255-2264 (1994); Synthetic Communications 28(10), 1743-1753 (1998) or Tetrahedron Letters 27(23), 2567-70 (1986)]. β-Oxoacetals of the formula IVa are likewise known, for example from EP 259139, or they are commercially available.

Compounds of the formula II in which Q is Q-2 or Q-3 and R¹ is hydrogen can be prepared by the synthesis route shown in Scheme 3:

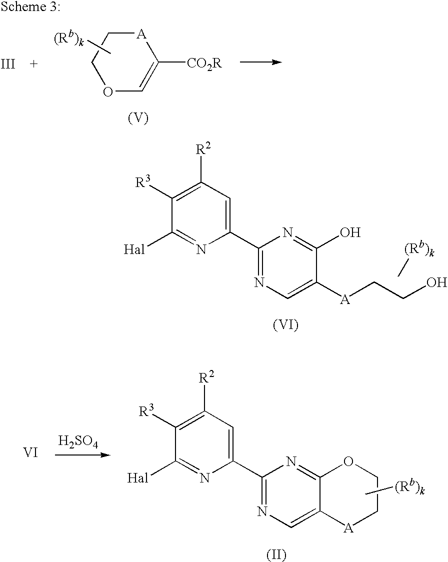

In Scheme 3, Hal, k, R^{b}, R² and R³ are as defined above. A is CH₂ or a chemical bond. R is C₁-C₄-alkyl, in particular methyl or ethyl. According to Scheme 3, the amidine compound III is, at 60-90° C. and in the presence of a base, for example an alkali metal alkoxide, such as sodium methoxide or sodium ethoxide in methanol or ethanol, reacted with the ester of the formula V. If the amidine III is not employed as hydrochloride, the addition of the base may be dispensed with (Liebigs Ann. Chem. 1974, pp. 468-476). The bishydroxy compound of the formula VI obtained in this manner is then subjected to a cyclizing dehydration, for example by treatment with sulfuric acid. The esters of the formula V are known or can be prepared analogously to processes known from the literature (see J. Heterocycl. Chem., 32 (1995) p. 735 and Liebigs Ann. Chem. 1974, pp. 468-476).

For their part, the compounds of the general formula III can be prepared from the corresponding 2-cyanopyridine compounds of the general formula VII (see Scheme 4). To this end, the 2-cyanopyridine compound VII is, using the method described in U.S. Pat. No. 4,873,248, converted by successive treatment with alkali metal alkoxide, such as sodium methoxide or ethoxide, and subsequent reaction with ammonium chloride, into the compound III. Instead of the hydrochlorides, it is also possible to use the hydrobromides, acetates, sulfates or formates in the subsequent steps shown in Schemes 1 to 3. The cyanopyridines of the formula VII are known, for example from US 2003/087940, WO 2004/026305, WO 01/057046 and Bioorg. Med. Chem. Lett. pp. 1571-1574 (2003), or they can be prepared by known preparation processes.

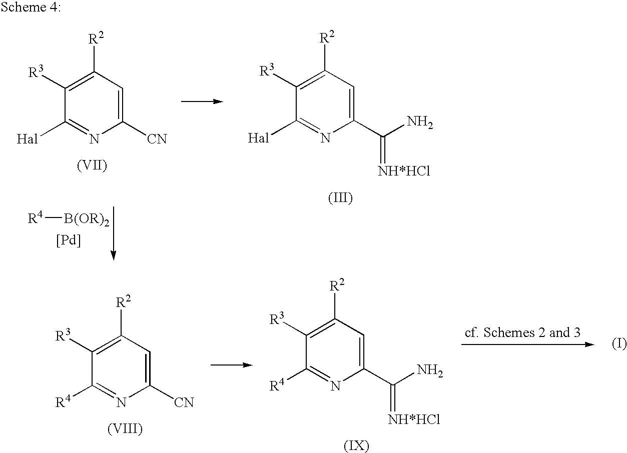

According to a second synthesis route (see Scheme 4), the compounds according to the invention can be prepared from the cyanopyridines VII. To this end, the compound VII is initially coupled with the (het)arylboronic acid compound R⁴—B(OR)₂, as described for Scheme 1, and the resulting 6-(het)aryl-2-cyanopyridine is converted under the reaction conditions described for compounds VII into the amidine compound IX. Compound IX can then be converted under the conditions mentioned for Schemes 2 and 3 into the corresponding compound of the formula I.

Compound of the general formula VII can, if they are not known, be prepared in particular by the process shown in Scheme 5.

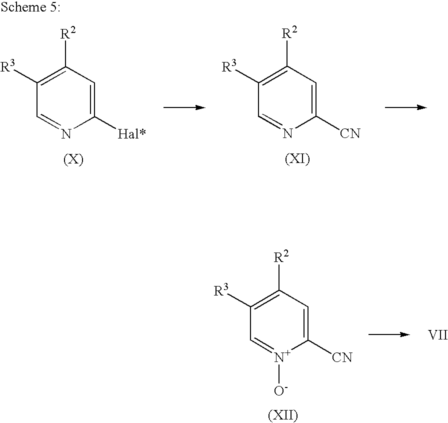

In Scheme 5, R² and R³ are as defined above. Hal* is chlorine, bromine or iodine.

The conversion of the 2-halopyridine X into the 2-cyanopyridine XI is performed using standard methods of organic chemistry by reacting X with cyanide ions, for example with sodium or potassium cyanide (see EP-A 97460, preparation example 1), copper(1) cyanide (see EP-A 34917, preparation example 3) or trimethylsilyl cyanide. The compound XI obtained in this manner is then converted by treatment with a peracid using methods known per se into the pyridine N-oxide XII. The conversion of XI into XII may be carried out analogously to known processes, for example by treating XI with hydrogen peroxide in an organic acid such as formic acid, acetic acid, chloroacetic acid or trifluoroacetic acid (see, for example, J. Org. Chem. 55, pp. 738-741 (1990) and Organic Synthesis, Collect. Vol. IV, pp. 655-656 (1963)) or by reacting XI with an organic peracid, such as meta-chloroperbenzoic acid, in an inert solvent, for example a halogenated hydrocarbon, such as dichloromethane or dichloroethane (see, for example, Synthetic Commun. 22(18), p. 2645, (1992); J. Med. Chem. 2146 (1998)). The conversion of XI into XII can also be carried out analogously to the method described by K. B. Sharpless (J. Org. Chem. 63(5), p. 7740 (1998)) by reacting XI in a halogenated hydrocarbon, such as dichloromethane or dichloroethane, in the presence of catalytic amounts (for example 5% by weight) of rhenium(VII) compounds, such as methyltrioxorhenium (H₃CR^{e}O₃), with hydrogen peroxide.

XII is then reacted with a halogenating agent, such as POCl₃ or POBr₃, which yields the corresponding compound VII. For the conversion of XII into VII the halogenating agent is generally employed in excess, based on the stoichiometry of the reaction. The reaction can be carried out in an inert organic solvent and is frequently carried out in the absence of a solvent, the halogenating agent then generally acting as solvent. The reaction temperature is usually in the range of from 20° C. to the boiling point of the halogenating agent. If appropriate, it is advantageous initially to introduce a chlorine atom into the 2-position of the pyridine N-oxide XII using a chlorinating agent such as POCl₃ and then to carry out a halogen exchange, for example by treatment with HBr or an iodinating agent, giving a compound of the formula VII where Hal=Br or I.

Compounds of the formula II in which Q is a radical Q-5 or Q-7 and R¹ is hydrogen can, according to the synthesis route shown in scheme 6, be furthermore prepared from 2-cyanopyridine compounds of the formula VII:

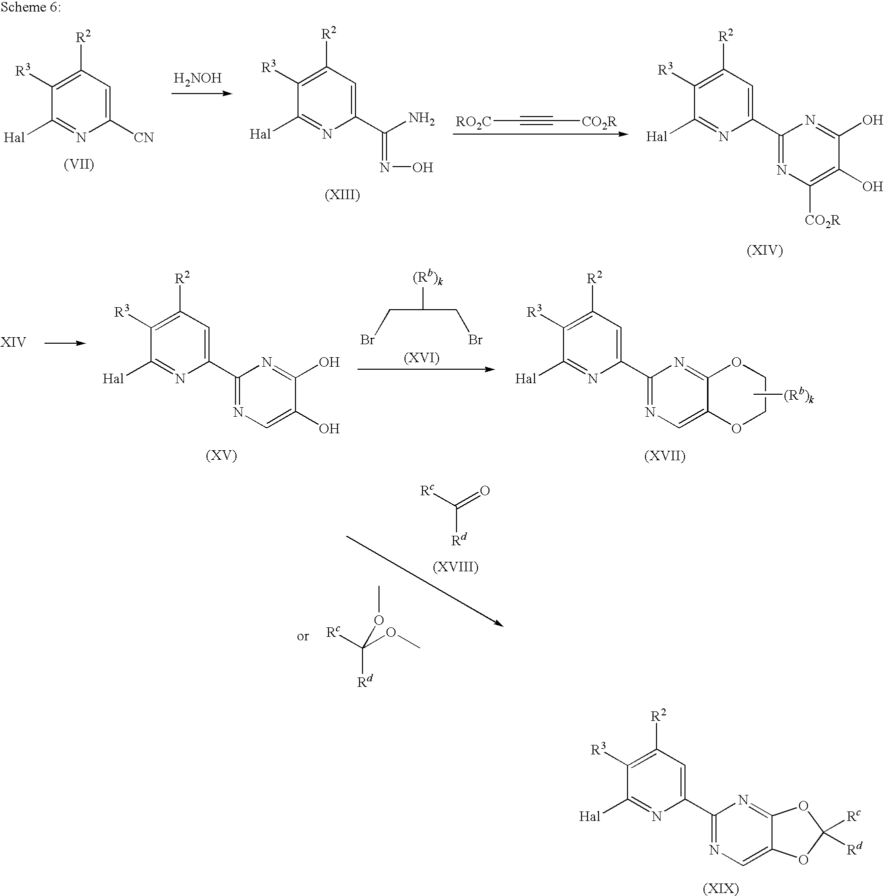

In Scheme 6, R², R³, R^{b} and k are as defined above. R is C₁-C₄-alkyl, in particular methyl. R^{c} and R^{d} independently of one another are hydrogen or C₁-C₄-alkyl.

In a first step, the 2-halo-6-cyanopyridine VII is reacted with hydroxylamine or with hydroxylamine hydrochloride in the presence of a base, such as potassium carbonate.

The reaction can be carried out analogously to the reactions in Farmaco, Ed. Sci., 41 (1986) p. 499. The N-hydroxyamidine XIII is then reacted with an acetylenedicarboxylic ester, which gives the 2-(2-halopyridin-6-yl)-4,5-bishydroxy-6-alkoxycarbonylpyrimidine XIV. The reaction of XIII with the acetylenedicarboxylic ester can be carried out analogously to the reaction in J. Heterocycl. Chem. 16 (1979) 1423. Compound XIV is then subjected to alkaline hydrolysis, for example by treatment with sodium hydroxide or potassium hydroxide and subsequently decarboxylated by treatment with aqueous acid, for example by treatment with dilute hydrochloric acid, which gives the 2-(2-halopyridin-6-yl)-4,5-bishydroxypyrimidine XV. The bishydroxypyrimidine XV obtained in this manner can then be reacted with an 1,2-dibromoalkane XVI, preferably in the presence of a base, such as an alkali metal hydroxide or alkali metal alkoxide, analogously to the method described in Heterocycl. Chem. 27, p. 151 (1990), which yields the fused pyrimidine XVII. Moreover, the bishydroxypyrimidine XV can be reacted analogously to the method described in Chem. Berichte 124 (3) 481 (1991); J. Chem. Soc., Perkin Trans 1 p. 3561 (1998); Synthesis p. 122 (1986) with a ketone or an aldehyde XVIII, which affords the fused pyrimidine XIX.

A further access to the compounds of the general formula II is illustrated in Scheme 7.

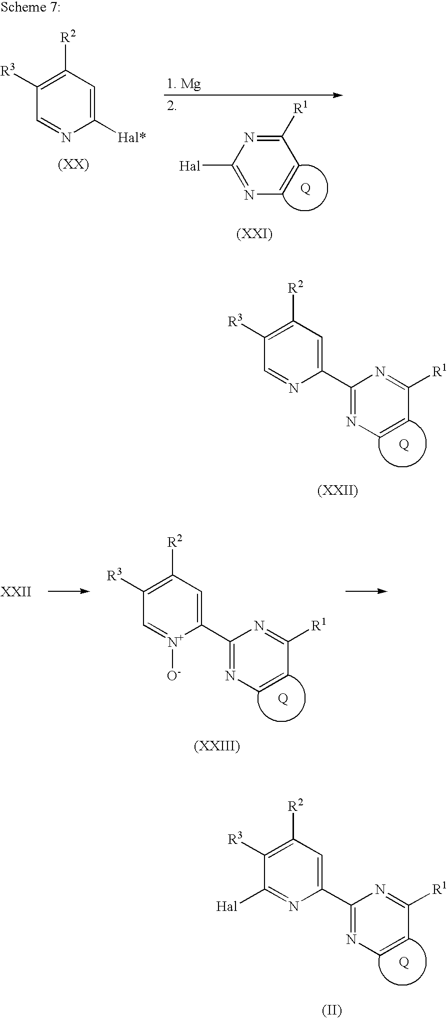

In Scheme 7, R¹, R², R³ and Q are as defined above. Hal* is chlorine, bromine or in particular iodine. Hal* is chlorine or bromine.

According to Scheme 7, the halopyridine of the formula XX is initially, by reaction with magnesium, converted into the corresponding Grignard compound which is then coupled with the 2-halopyrimidine compound XXI. The Grignard compound can be prepared by processes known per se as described, for example, in Synlett p. 1359 (1998). Subsequent coupling with the 2-halopyrimidine compound XXI is usually carried out in the presence of a transition metal catalyst, a metal of groups 8 to 10 (according to IUPAC 1989), in particular a palladium, nickel or iron catalyst. In this context, reference is made to the catalysts mentioned above. The reaction is carried out in a solvent suitable for this purpose, for example an ether, such as diethyl ether, dioxane, tetrahydrofuran, an aromatic hydrocarbon, such as toluene or xylene, or an aprotic amide, lactam or urea, such as N-methylpyrrolidone or dimethylpropyleneurea, or in mixtures of these solvents, in particular mixtures comprising at least one ether. The reaction temperatures are generally in the range from −40 to +120° C. and in particular in the range from 20 to 100° C. For further details, reference is made to the methods described in J. Am. Chem. Soc. 124, p. 13856 (2002), Chem. Pharm. Bull., p. 4533 (1983) and Chem. Pharm. Bull., p. 2005 (1984), which can be applied in a manner analogous to the coupling of XX with XXI.

The compound XXII obtained in this manner is then converted into the N-oxide XXIII. The conversion of XXII into the 2-phenylpyridine N-oxide of the formula XXIII can be carried out analogously to known processes, for example by treating XXII with hydrogen peroxide in an organic acid, such as formic acid, acetic acid, chloroacetic acid or trifluoroacetic acid (see, for example, J. Org. Chem. 55, pp. 738-741 (1990) and Organic Synthesis, Collect. Vol. IV, pp. 655-656 (1963)) or by reacting XXII with an organic peracid, such as meta-chloroperbenzoic acid, in an inert solvent, for example a halogenated hydrocarbon, such as dichloromethane or dichloroethane (see, for example, Synthetic Commun. 22(18), p. 2645, (1992); J. Med. Chem. 2146 (1998)). The conversion of XXII into XXIII can also be carried out analogously to the method described by K. B. Sharpless (J. Org. Chem. 63(5), p. 7740 (1998)) by reacting XXII in a halogenated hydrocarbon, such as dichloromethane or dichloroethane, in the presence of catalytic amounts (for example 5% by weight) of rhenium(VII) compounds, such as methyltrioxorhenium (H₃CR^{e}O₃), with hydrogen peroxide.

Analogously to the conversion of XI into XII shown in Scheme 5, the N-oxide XXIII is then reacted with a halogenating agent, such as POCl₃ or POBr₃, and a halogen exchange is carried out, if appropriate, giving the 2-halo compound II. According to the method given in Scheme 1 this is then reacted with a (het)arylboronic acid compound of the corresponding Grignard compound, giving the compound of the general formula I.

Compounds of the general formula XX are known or can be prepared by methods of organic chemistry which are known per se (see, for example, U.S. Pat. No. 6,040,448, WO 99/21850 and Chem. Pharm. Bull p. 2254 (1983)).

The reaction mixtures obtained by the processes shown in Schemes 1 to 7 are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally however not necessarily required since in some cases the individual isomers can be interconverted during work-up for use or during application (for example under the action of light, acids or bases). Such conversions may also take place after use, for example, in the case of treatment of plants, in the treated plant, or in the harmful fungus to be controlled.

The compounds of the formula I are suitable as fungicides. They are distinguished by excellent activity against a broad spectrum of phytopathogenic fungi from the classes of the Ascomycetes, Deuteromycetes, Oomycetes and Basidiomycetes, in particular from the class of the Oomycetes. Some of them are systemically active and can be used in crop protection as foliar fungicides, as fungicides for seed dressing and as soil fungicides.

They are particularly important in the control of a large number of fungi on various crop plants, such as wheat, rye, barley, oats, rice, corn, grass, bananas, cotton, soybeans, coffee, sugar cane, grapevines, fruit and ornamental plants and vegetable plants, such as cucumbers, beans, tomatoes, potatoes and cucurbits, and also on the seeds of these plants.

They are especially suitable for controlling the following plant diseases:
Alternaria species on vegetables, oilseed rape, sugar beet, fruit, rice, soybeans, and also on potatoes (for example A. solani or A. alternata) and tomatoes (for example A. solani or A. alternata) and Alternaria ssp. (black head) on wheat, Aphanomyces species on sugar beet and vegetables, Ascochyta species on cereals and vegetables, for example Ascochyta tritici (leaf spot) on wheat,
Bipolaris and Drechslera species on corn, cereals, rice and lawn (for example D. maydis on corn, D. teres on barley, D. tritici-repentis on wheat),
Blumeria graminis (powdery mildew) on cereals (for example wheat or barley),
Botrytis cinerea (gray mold) on strawberries, vegetables, flowers, grapevines and wheat,
Bremia lactucae on lettuce,
Cercospora species on corn, soybeans, rice and sugar beet and, for example, Cercospora sojina (leaf blotch) or Cercospora kikuchii (leaf blotch) on soybeans, Cladosporium herbarum (black mold) on wheat,
Cochliobolus species on corn, cereals and rice (for example Cochliobolus sativus on cereals and Cochliobolus miyabeanus on rice),
Colletotricum species on soybeans, cotton and other plants (for example C. acutatum on various plants and, for example, Colletotrichum truncatum (antracnose) on soybeans),
Corynespora cassiicola (leaf blotch) on soybeans,
Dematophora necatrix (root/stem rot) on soybeans,
Diaporthe phaseolorum (stem disease) on soybeans,
Drechslera species, Pyrenophora species on corn, cereals, rice and lawn, on barley (for example D. teres) and on wheat (for example D. tritici-repentis),
Esca on grapevines, caused by Phaeoacremonium chlamydosporium, Ph. Aleophilum, and Formitipora punctata (syn. Phellinus punctatus), Elsinoe ampelina on grapevines,
Epicoccum spp. (black head) on wheat,
Exserohilum species on corn,
Erysiphe cichoracearum and Sphaerotheca fuliginea on cucumbers,
Fusarium and Verticillium species (for example V. dahliae) on various plants: for example F. graminearum or F. culmorum (root rot) on cereals (for example wheat or barley) or, for example, F. oxysporum on tomatoes and Fusarium solani (stem disease) on soybeans,
Gaeumanomyces graminis on cereals (for example wheat or barley),
Gibberella species on cereals and rice (for example Gibberella fujikuroi on rice),
Glomerella cingulata on grapevines and other plants,
Grainstaining complex on rice,
Guignardia budwelli on grapevines,
Helminthosporium species (for example H. graminicola) on corn and rice,
Isariopsis clavispora on grapevines,
Macrophomina phaseolina (root/stem rot) on soybeans,
Michrodochium nivale on cereals (for example wheat or barley),
Microsphaera diffusa (powdery mildew) on soybeans,
Mycosphaerella species on cereals, bananas and peanuts (M. graminicola on wheat, M. fijiensis on bananas),
Peronospora species on cabbage (for example P. brassicae), bulbous plants (for example P. destructor) and, for example, Peronospora manshurica (downy mildew) on soybeans,
Phakopsara pachyrhizi and Phakopsara meibomiae on soybeans,
Phialophora gregata (stem disease) on soybeans,
Phomopsis species on soybeans, sunflowers and grapevines (P. viticola on grapevines, P. helianthii on sunflowers),
Phytophthora species on various plants, for example P. capsici on bell peppers, Phytophthora megasperma (leaf/stem rot) on soybeans, Phytophthora infestans on potatoes and tomatoes,
Plasmopara viticola on grapevines,
Podosphaera leucotricha on apples,
Pseudocercosporella herpotrichoides on cereals,
Pseudoperonospora species on hops and cucumbers (for example P. cubensis on cucumbers or P. humili on hops),
Pseudopezicula tracheiphilai on grapevines,
Puccinia species on various plants, for example P. triticina, P. striformins, P. hordei or P. graminis on cereals (for example wheat or barley) or on asparagus (for example P. asparagi),
Pyrenophora species on cereals,
Pyricularia oryzae, Corticium sasakii, Sarocladium oryzae, S. attenuatum, Entyloma oryzae on rice,
Pyricularia grisea on lawn and cereals,
Pythium spp. on lawn, rice, corn, cotton, oilseed rape, sunflowers, sugar beet, vegetables and other plants (for example P. ultimum or P. aphanidermatum),
Ramularia collo-cygni (physiological leaf spots) on barley,
Rhizoctonia species (for example R. solani) on cotton, rice, potatoes, lawn, corn, oilseed rape, potatoes, sugar beet, vegetables and other plants, for example Rhizoctonia solani (root/stem rot) on soybeans or Rhizoctonia cerealis (yellow patch) on wheat or barley,
Rhynchosporium secalis on barley (scald), rye and triticale,
Sclerotinia species on oilseed rape, sunflowers and other plants, for example Sclerotinia sclerotiorum (stem disease) or Sclerotinia rolfsii (stem disease) on soybeans,
Septoria glycines (brown spot) on soybeans,
Septoria tritici and Stagonospora nodorum on wheat,
Erysiphe (syn. Uncinula) necator on grapevines,
Setospaeria species on corn and lawn,
Sphacelotheca reilinia on corn,
Stagonospora nodorum (glume blotch) on wheat,
Thievaliopsis species on soybeans and cotton,
Tilletia species on cereals,
Typhula incarnata (snow mold) on wheat or barley,
Ustilago species on cereals, corn and sugar beet,
Venturia species (scab) on apples and pears (for example V. inaequalis on apples).

The compounds of the formula I are furthermore suitable for controlling harmful fungi in the protection of materials (for example wood, paper, paint dispersions, fibers or fabrics) and in the protection of stored products. In the protection of wood, particular attention is paid to the following harmful fungi:

Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds of the formula I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment amounts of active compound of from 1 to 1000 g/100 kg, preferably from 5 to 100 g/100 kg, of seed are generally necessary.

When used in the protection of materials or stored products, the amount of active compound applied depends on the kind of application area and on the desired effect.

Amounts customarily applied in the protection of materials are, for example, 0.001 g to 2 kg, preferably 0.005 g to 1 kg, of active compound per cubic meter of treated material.

The compounds of the formula I can be present in different crystal modifications which may differ in their biological activity. They also form part of the subject matter of the present invention.

The compounds of the formula. I can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol; ethanol, propanol, butanol, cyclohexanol, cyclohexanone, isophorone, highly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations:

1. Products for Dilution with Water

### A Water-Soluble Concentrates (SL, LS)

10 parts by weight of the active compounds are dissolved in 90 parts by weight of water or in a water-soluble solvent. As an alternative, wetters or other auxiliaries are added. The active compound dissolves upon dilution with water. In this way, a formulation having a content of 10% by weight of active compound is obtained.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (e.g. Ultraturax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetters and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations

In a ball mill, 20 parts by weight of the active compounds, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

### 2. Products to be Applied Undiluted

I Dustable powders (DP, DS)

5 parts by weight of the active compounds are ground finely and mixed intimately with 95 parts by weight of finely divided kaolin. This gives a dustable product having an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules to be applied undiluted having an active compound content of 0.5% by weight.

K ULV solutions (UL)

10 parts by weight of the active compounds are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

Aqueous use forms can be prepared from emulsion concentrates, pastes or wettable powders (wettable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra-low volume (ULV) process, it being possible to apply formulations with more than 95% by weight of active compound or even to apply the active compound without additives.

Oils of various types, wetting agents, adjuvants, herbicides, fungicides, other pesticides, or bactericides may be added to the active compounds, even, if appropriate, not until immediately prior to use (tank mix). These agents may be admixed with the compositions according to the invention in a weight ratio of from 1:100 to 100:1, preferably from 1:10 to 10:1.

Suitable adjuvants in this sense are in particular: organically modified polysiloxanes, for example Break Thru S 2400; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the use form as fungicides, also be present together with other active compounds, for example with herbicides, insecticides, growth regulators, fungicides or also with fertilizers. By mixing the compounds I or the compositions comprising them in the use form as fungicides with other fungicides, in many cases a broadening of the spectrum of fungicidal activity is achieved.

The following list of fungicides, with which the compounds according to the invention can be used in conjunction, is intended to illustrate the possible combinations without being limited thereto:

strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methyl-pyridin-2-ylmethoxyimino)ethyl]benzyl)carbamate, methyl 2-(ortho-(2,5-dimethylphenyl-oxymethylene)phenyl)-3-methoxyacrylate;

carboxamides
carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chloro-phenyl)prop-2-ynyloxy]-3-methoxyphenyl)ethyl)-2-methanesulfonylamino-3-methyl-butyramide, N-(2-(4-[3-(4-chlorophenyl)prop-2-ynyloxy]-3-methoxyphenyl)ethyl)-2-ethanesulfonylamino-3-methylbutyramide;
   azoles
triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;
   nitrogenous Heterocyclyl Compounds
pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propyl-chromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### PREPARATION EXAMPLES

### Example 18

### 2-(5′-Methoxy-3-methyl-[2,2]-bipyridinyl-6-yl)-5,6,7,8-tetrahydroquinazoline

### 18.1 6-Bromo-5-methylpyridin-2-carboxamidine hydrochloride

2.2 g of a 30% strength sodium methoxide solution in methanol were added to 4.90 g (25 mmol) of 6-bromo-5-methylpyridine-2-carbonitrile (for the preparation, see US 2003/0087940 A1 and Bioorg. Med. Chem. Lett. 1571-1574 (2003)] in 60 ml methanol, and the mixture was stirred at 23° C. for 7 hours. 1.5 g of ammonium chloride were then added, and the mixture was stirred at 23° C. for a further 8 hours. After removal of the solvent, methyl tert-butyl ether was added and the product was filtered off with suction, which gave 4.2 g of a white solid which was used without purification for the next step.

### 18.2 2-(6-Bromo-5-methyl-pyridin-2-yl)-5,6,7,8-tetrahydroquinazoline

3.6 g of sodium methoxide (30% strength solution in methanol) were added to a solution of 4.2 g (7 mmol) of 6-bromo-5-methylpyridine-2-carboxamidine hydrochloride in 100 ml of methanol. After 30 min, 3.1 g (20 mmol) of 2-dimethylaminomethylene-cyclohexanone [for the preparation, see US 2004132708; European Journal of Organic Chemistry 10, 2485 (1999) and Synthetic Communications 28(10), 1743 (1998)] were added and the mixture was stirred under reflux for 2 hours. The reaction solution was then partitioned between water and methyl tert-butyl ether. The organic phase was separated off, the solvent was removed under reduced pressure and the residue was chromatographed on silica gel using cyclohexane/methyl tert-butyl ether (1:1), which gave 2.2 g of the title compound.

¹H-NMR (δ, CDCl₃,): 1.7-1.8 (m); 2.4 (s); 2.7 (m); 3.0 (m); 7.6 (m); 8.3 (m); 8.7 (s).

### 18.3 2-(5′-Methoxy-3-methyl-[2,2]-bipyridinyl-6-yl)-5,6,7,8-tetrahydroquinazoline

0.24 g of 2-methoxy-5-pyridineboronic acid and 0.26 g of sodium carbonate in 12 ml of water were added successively to a solution of 0.3 g of 2-(6-bromo-5-methylpyridin-2-yl)-5,6,7,8-tetrahydroquinazoline in 16 ml of dimethoxyethane. After addition of about 30 mg of [1,4-bis(diphenylphosphino)butane]palladium(II) dichloride, the mixture was heated under reflux for 4 hours. The reaction solution was then partitioned between water and methyl tert-butyl ether. The organic phase was separated off, the solvent was removed under reduced pressure and the residue was chromatographed on silica gel using cyclohexane/methyl tert-butyl ether (3:1) and methyl tert-butyl ether/ethanol (9:1). The product was then triturated with methyl tert-butyl ether and pentane. This gave 0.16 g of the title compound of melting point 157-158° C.

### Example 39

### 2-(3-Methyl-[2,4′]-bipyridinyl-6-yl)-,6,7,8,9-tetrahydro-5H-cyclohepta-pyrimidine

### 39.1 2-(6-Bromo-5-methylpyridin-2-yl)-6,7,8,9-tetrahydro-5H-cycloheptapyrimidine

51.7 g of sodium methoxide (30% strength solution in methanol) were added to a solution of 30 g (120 mmol) of 6-bromo-5-methylpyridine-2-carboxamidine hydrochloride from 18.1 in 300 ml of methanol. After 30 min, 30 g (180 mmol) of 2-dimethylaminomethylenecycloheptanone [for the preparation, see Tetrahedron 50(7), 2255-64 (1994); Synthetic Communications 28(10), 1743-1753 (1998) and Tetrahedron Letters 27(23), 2567-70 (1986)] were added, and the mixture was stirred under reflux for 5 hours. The solvent from the reaction solution was then removed under reduced pressure and the residue was chromatographed on silica gel using cyclohexane/methyl tert-butyl ether (3:1) and methyl tert-butyl ether/EtOH (9:1). The product obtained was then triturated with 250 ml of n-pentane and a little methyl tert-butyl ether for 30 minutes. This gave 20 g of the title compound.

¹H-NMR (δ, CDCl₃,): 2.5 (s); 2.8 (m); 3.1 (m); 7.6 (m); 8.3 (m); 8.5 (s).

### 39.2 Preparation of 2-(3-methyl-[2,4′]-bipyridinyl-6-yl)-6,7,8,9-tetrahydro-5H-cycloheptapyrimidine

0.17 g of 4-pyridineboronic acid and 0.25 g of sodium carbonate in 12 ml of water were added successively to a solution of 0.3 g 2-(6-bromo-5-methyl-pyridin-2-yl)-6,7,8,9-tetrahydro-5H-cycloheptapyrimidin in 16 ml dimethoxyethane. After addition of about 30 mg of [1,4-bis(diphenylphosphino)butane]palladium(II) dichloride, the mixture was stirred under reflux for 4 hours. The reaction solution was then partitioned between water and methyl tert-butyl ether. The organic phase was separated off, the solvent was removed under reduced pressure and the residue was chromatographed on silica gel using methyl tert-butyl ether/ethanol (4:1), which gave 0.15 g of the title compound of melting point 198 to 200° C.

### Example 80

### 2-[6-(2,3-Difluorophenyl)-5-methylpyridin-2-yl)]-7,8-dihydro-5H-pyrano[4,3-d]-pyrimidine

### 80.1 Preparation of 2-(6-bromo-5-methylpyridin-2-yl)-7,8-dihydro-5H-pyrano[4,3-d]-pyrimidine

8.6 g of sodium methoxide (30% strength solution in methanol) were added to a solution of 5 g (20 mmol) of 6-bromo-5-methylpyridine-2-carboxamidine hydrochloride from Example 18.1 in 60 ml of methanol. After 30 min, 4.7 g (30 mmol) of 3-dimethylaminomethylenetetrahydropyran-4-one [for the preparation, see WO 2004/060890 and Journal of Heterocycl. Chem. 21 (5), 1441 (1984)] were added, and the mixture was stirred under reflux for 5 hours. The solvent from the reaction solution was then removed under reduced pressure and the residue was chromatographed on silica gel using cyclohexane/methyl tert-butyl ether (1:1) and methyl tert-butyl ether. This gave 1.4 g of the title compound as a yellowish solid.

¹H-NMR (δ, CDCl₃,): 2.5 (s); 3.1 (m); 4.1 (m); 4.7 (s); 7.65 (m); 8.35 (m) and 8.6 (s).

### 80.2 Preparation of 2-[6-(2,3-difluorophenyl)-5-methylpyridin-2-yl)]-7,8-dihydro-5H-pyrano[4,3-d]-pyrimidine

0.25 g of 2,3-difluorophenylboronic acid and 0.41 g of sodium carbonate in 20 ml of water were added successively to a solution of 0.4 g of 2-(6-bromo-5-methylpyridin-2-yl)-7,8-dihydro-5H-pyrano[4,3-d]-pyrimidine from Example 80.1 in 20 ml of ethylene glycol dimethyl ether. After addition of about 30 mg of [1,4-bis(diphenylphosphino)-butane]palladium(II) dichloride, the mixture was stirred under reflux for 5 hours. The reaction solution was then partitioned between water and methyl tert-butyl ether. The organic phase was separated off, the solvent was removed under reduced pressure and the residue was chromatographed on silica gel using cyclohexane/methyl tert-butyl ether (1:1), which gave 0.25 g of the title compound of melting point 160 to 163° C.

The compounds I listed in Tables 1 and 2 and the compounds I of Examples 81 to 89 were prepared in an analogous manner.

TABLE 1

| | |
|---|---|
| | (I) |
| 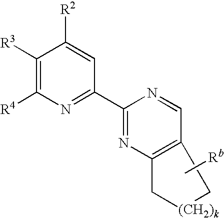 | |

| | | | | | | m.p. (°C.)/ |
|---|---|---|---|---|---|---|
| Ex. | R⁴ | R³ | R² | R^{b} | k | consistency |
| 1 | 3-furyl | CH₃ | H | H | 1 | 194-198 |
| 2 | thien-2-yl | CH₃ | H | H | 1 | 194-198 |
| 3 | thien-3-yl | CH₃ | H | H | 1 | 163-167 |
| 4 | 3-methylthien-2-yl | CH₃ | H | H | 1 | 108-112 |
| 5 | 4-methylthien-2-yl | CH₃ | H | H | 1 | 197-199 |
| 6 | 5-methylthien-2-yl | CH₃ | H | H | 1 | 132-137 |
| 7 | 5-chlorothien-2-yl | CH₃ | H | H | 1 | 162-164 |
| 8 | 4-methylthiophen-3-yl | CH₃ | H | H | 1 | 171-174 |
| 9 | 5-acetylthien-2-yl | CH₃ | H | H | 1 | oil |
| 10 | 5-methoxyiminoethyl- | CH₃ | H | H | 1 | 188-195 |
| | thien-2-yl | | | | | |
| 11 | pyridin-3-yl | CH₃ | H | H | 1 | 210-212 |
| 12 | 2-chloropyridin-3-yl | CH₃ | H | H | 1 | 207-209 |
| 13 | 6-chloropyridin-3-yl | CH₃ | H | H | 1 | 192-194 |
| 14 | 6-fluoropyridin-3-yl | CH₃ | H | H | 1 | 139-143 |
| 15 | 6-methoxypyridin-3-yl | CH₃ | H | H | 1 | 165-167 |
| 16 | pyridin-4-yl | CH₃ | H | H | 1 | 183-185 |
| 17 | pyrimidin-5-yl | CH₃ | H | H | 1 | 177 |
| 18 | 2-methoxypyridin-5-yl | CH₃ | H | H | 2 | 157-158 |
| 19 | pyridin-3-yl | CH₃ | H | H | 2 | 165-168 |
| 20 | pyridin-4-yl | CH₃ | H | H | 2 | 197-201 |
| 21 | 2-chloropyridin-3-yl | CH₃ | H | H | 2 | 183-186 |
| 22 | 6-chloropyridin-3-yl | CH₃ | H | H | 2 | 170-173 |
| 23 | 6-fluoropyridin-3-yl | CH₃ | H | H | 2 | 158-160 |
| 24 | 6-methoxypyridin-3-yl | CH₃ | H | H | 2 | 157-158 |
| 25 | pyrimidin-5-yl | CH₃ | H | H | 2 | 159-162 |
| 26 | 3-furyl | CH₃ | H | H | 2 | 156-158 |
| 27 | thien-2-yl | CH₃ | H | H | 2 | 148-151 |
| 28 | thien-3-yl | CH₃ | H | H | 2 | 145-148 |
| 29 | 3-methylthien-2-yl | CH₃ | H | H | 2 | oil |
| 30 | 4-methylthien-2-yl | CH₃ | H | H | 2 | 173-188 |
| 31 | 5-methylthien-2-yl | CH₃ | H | H | 2 | 88-93 |
| 32 | 4-methylthien-3-yl | CH₃ | H | H | 2 | 169-173 |
| 33 | 5-chlorothien-2-yl | CH₃ | H | H | 2 | 140-143 |
| 34 | 5-acetylthien-2-yl | CH₃ | H | H | 2 | 197-199 |
| 35 | 5-methoxyiminoethyl- | CH₃ | H | H | 2 | 156-166 |
| | thien-2-yl | | | | | |
| 36 | 5-ethoxyiminoethyl- | CH₃ | H | H | 2 | 115-119 |
| | thien-2-yl | | | | | |
| 37 | 5-n-hexoxyiminoethyl- | CH₃ | H | H | 2 | 82-85 |
| | thien-2-yl | | | | | |
| 38 | pyridin-3-yl | CH₃ | H | H | 3 | 148-151 |
| 39 | pyridin-4-yl | CH₃ | H | H | 3 | 198-200 |
| 40 | pyrimidin-5-yl | CH₃ | H | H | 3 | 163-165 |
| 41 | 2-chloropyridin-3-yl | CH₃ | H | H | 3 | 197-199 |
| 42 | 6-chloropyridin-3-yl | CH₃ | H | H | 3 | 182-185 |
| 43 | 6-fluoropyridin-3-yl | CH₃ | H | H | 3 | 157-159 |
| 44 | 6-methoxypyridin-3-yl | CH₃ | H | H | 3 | 134-137 |
| 45 | 3-furyl | CH₃ | H | H | 3 | 144-147 |
| 46 | thien-2-yl | CH₃ | H | H | 3 | 130-133 |
| 47 | thien-3-yl | CH₃ | H | H | 3 | 156-158 |
| 48 | 3-methylthien-2-yl | CH₃ | H | H | 3 | oil |
| 49 | 4-methylthien-2-yl | CH₃ | H | H | 3 | 143-144 |
| 50 | 4-methylthien-3-yl | CH₃ | H | H | 3 | 137-138 |
| 51 | 5-chlorothien-2-yl | CH₃ | H | H | 3 | 144-147 |
| 52 | 5-acetylthien-2-yl | CH₃ | H | H | 3 | 176-177 |
| 53 | 5-methoxyiminoethyl- | CH₃ | H | H | 3 | 168-171 |
| | thien-2-yl | | | | | |
| 54 | 5-ethoxyiminoethyl- | CH₃ | H | H | 3 | 131-133 |
| | thien-2-yl | | | | | |
| 55 | 5-n-hexoxyiminoethyl- | CH₃ | H | H | 3 | 72-75 |
| | thien-2-yl | | | | | |
| 56 | pyridin-3-yl | CH₃ | H | 6,6-(CH₃)₂ | 2 | 198 |
| 57 | pyridin-4-yl | CH₃ | H | 6,6-(CH₃)₂ | 2 | 180-181 |
| 58 | pyrimidin-5-yl | CH₃ | H | 6,6-(CH₃)₂ | 2 | 192-193 |
| 59 | 2-chloropyridin-3-yl | CH₃ | H | 6,6-(CH₃)₂ | 2 | 214-215 |
| 60 | 6-chloropyridin-3-yl | CH₃ | H | 6,6-(CH₃)₂ | 2 | 201-206 |
| 61 | 6-fluoropyridin-3-yl | CH₃ | H | 6,6-(CH₃)₂ | 2 | 143-146 |
| 62 | 6-methoxypyridin-3-yl | CH₃ | H | 6,6-(CH₃)₂ | 2 | 152-156 |
| 63 | 3-furyl | CH₃ | H | 6,6-(CH₃)₂ | 2 | 160-162 |
| 64 | thien-2-yl | CH₃ | H | 6,6-(CH₃)₂ | 2 | 145-146 |
| 65 | thien-3-yl | CH₃ | H | 6,6-(CH₃)₂ | 2 | 206 |
| 66 | 4-methylthien-3-yl | CH₃ | H | 6,6-(CH₃)₂ | 2 | 186-187 |
| 67 | 3-methylthien-2-yl | CH₃ | H | 6,6-(CH₃)₂ | 2 | oil |

Ex. Example
m.p. melting point

TABLE 2

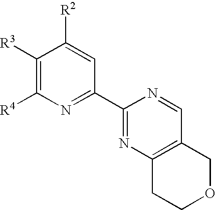

| Ex. | R⁴ | R³ | R² | m.p. (°C.) |
|---|---|---|---|---|
| 68 | pyridin-3-yl | CH₃ | H | 141 |
| 69 | pyridin-4-yl | CH₃ | H | 142 |
| 70 | 6-methoxypyridin-3-yl | CH₃ | H | 144 |
| 71 | 2-chloropyridin-3-yl | CH₃ | H | 146 |
| 72 | 6-chloropyridin-3-yl | CH₃ | H | 147 |
| 73 | 3-furyl | CH₃ | H | 148 |
| 74 | thien-2-yl | CH₃ | H | 149 |
| 75 | thien-3-yl | CH₃ | H | 150 |
| 76 | 4-methylthien-2-yl | CH₃ | H | 151 |
| 77 | 6-fluoropyridin-3-yl | CH₃ | H | 153 |
| 78 | 4-methylthien-3-yl | CH₃ | H | 154 |
| 79 | 3-methylthien-2-yl | CH₃ | H | 168 |
| 80 | 2,3-difluorophenyl | CH₃ | H | 160-163 |

Example 81: m.p. 177-188° C.

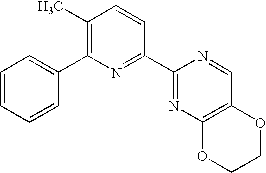

Example 82: m.p. 195-197° C.

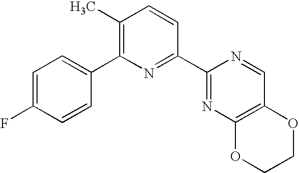

Example 83: M + H⁺: 320.10

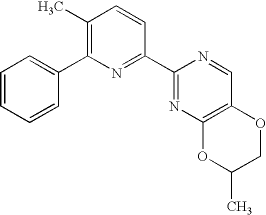

Example 84: m.p. 187-199° C.

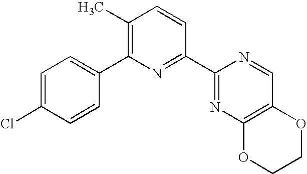

Example 85: M + H⁺: 334.10

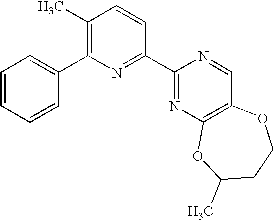

Example 86: m.p. 167.3-169.3° C.

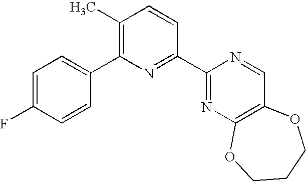

Example 87: M + H⁺: 352.10

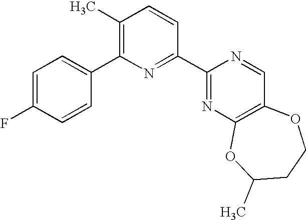

Example 88: m.p. 173-178° C.

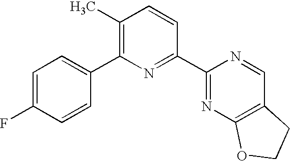

Example 89: m.p. 165-172° C.

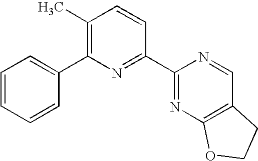

Ex. Example
m.p. melting point

### Test of the Fungicidal Activity:

The active compounds were prepared separately or together as a stock solution with 25 mg of active compound which was made up to 10 ml using a mixture of acetone and/or dimethyl sulfoxide (DMSO) and the emulsifier Wettoll® EM 31 (wetting agent having emulsifying and dispersing action based on the ethoxylated alkylphenols) in a volume ratio of solvent/emulsifier of 99 to 1. The mixture was then made up with water to 100 ml. This stock solution was diluted with the solvent/emulsifier/water mixture described to the concentration of active compound stated below.

### Use Example 1

### Activity Against Gray Mold on Bell Pepper Leaves Caused by Botrytis cinerea, 1 Day Protective Application

Bell pepper leaves of the cultivar “Neusiedler Ideal Elite” were, after 2 to 3 leaves were well developed, sprayed to runoff point with an aqueous suspension having the active compound concentration stated below. The next day, the treated plants were inoculated with a spore suspension of Botrytis cinerea which contained 1.7×10⁶ spores/ml in a 2% aqueous biomalt solution. The test plants were then placed in a dark climatized chamber at 22 to 24° C. and high atmospheric humidity. After 5 days, the extent of the fungal infection on the leaves could be determined visually in %.

In this test, the plants which had been treated with 250 ppm of active compounds from examples 1, 2, 3, 4, 5, 6, 8, 13, 14, 15, 19, 20, 21, 22, 23, 24, 26, 27, 28, 29, 31, 32, 34, 35, 36, 37, 38, 39, 41, 44, 45, 46, 47, 48, 49, 50 or 52 showed an infection of at most 20%, whereas the untreated plants were 100% infected.

### Use Example 2

### Activity Against Net Blotch of Barley Caused by Pyrenophora teres, 1 Day Protective Application

Leaves of potted barley seedlings were sprayed to runoff point with an aqueous suspension having the active compound concentration stated below. 24 hours after the spray coating had dried on, the test plants were inoculated with an aqueous spore suspension of Pyrenophora[syn. Drechslera] teres, the net blotch pathogen. The test plants were then placed in a greenhouse at temperatures between 20 and 24° C. and 95 to 100% relative atmospheric humidity. After 6 days, the extent of the development of the disease was determined visually in % infection of the entire leaf area.

In this test, the plants which had been treated with 250 ppm of active compounds from examples 1, 2, 3, 4, 6, 8, 12, 14, 15, 20, 23, 24, 27, 28, 29, 30, 31, 32, 34, 35, 36, 38, 39, 41, 42, 43, 44, 45, 46, 47, 48, 49, 50, 52, 53, 54, 55, 56, 59, 60, 65 or 67 showed an infection of at most 20%, whereas the untreated plants were 90% infected.

### Use Example 3

### Curative Activity Against Brown Rust of Wheat Caused by Puccinia recondita

Leaves of potted wheat seedlings of the cultivar “Kanzler” were inoculated with a spore suspension of brown rust of wheat (Puccinia recondite). The pots were then placed in a chamber with high atmospheric humidity (90 to 95%) and 20 to 22° C. for 24 hours. During this time, the spores germinated and the germ tubes penetrated into the leaf tissue. The next day, the infected plants were sprayed to runoff point with the above-described active compound solution having the active compound concentration stated below. After the spray coating had dried on, the test plants were cultivated in a greenhouse at temperatures between 20 and 22° C. and 65 to 70% relative atmospheric humidity for 7 days. The extent of the rust fungus development on the leaves was then determined.

In this test, the plants which had been treated with 250 ppm of active compound from example 44 showed no infection, whereas the untreated plants were 90% infected. The plants which, for comparison, had been treated under the same conditions with the compound from WO 2006/010570

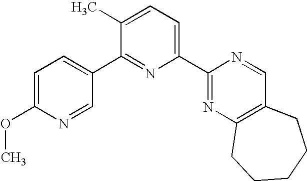

showed an infection of 90%.

1-16. (canceled)

17. A 2-(pyridin-2-yl)-pyrimidine compound of the general formula I 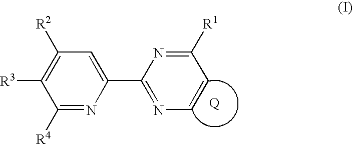
in which:
Q is a fused saturated 5-, 6- or 7-membered carbocycle or a 5-, 6- or 7-membered heterocycle which, in addition to the carbon ring members, has one or two heteroatoms selected from the group consisting of oxygen and sulfur as ring members, where the carbocycle and the heterocycle are unsubstituted or have 1, 2, 3 or 4 C₁-C₄-alkyl groups as substituents;
R¹ is hydrogen, OH, C₁-C₄-alkyl, C₁-C₄-alkoxy, C₁-C₄-haloalkyl, C₁-C₄-haloalkoxy or halogen;
R² is hydrogen, NO₂, halogen, C₁-C₆-alkyl, C₃-C₆-cycloalkyl, C₁-C₆-alkoxy, C₁-C₆-haloalkyl or C₁-C₆-haloalkoxy;
R³ is hydrogen, halogen, C₁-C₄-alkyl, C₁-C₄-alkoxy, C₁-C₄-haloalkyl or C₁-C₄-haloalkoxy;
R⁴ is phenyl, 5-membered heteroaryl which has 1, 2, 3 or 4 nitrogen atoms or 1 heteroatom selected from the group consisting of oxygen and sulfur and optionally 1, 2 or 3 nitrogen atoms as ring atoms, or 6-membered hetaryl which has 1, 2, 3 or 4 nitrogen atoms as ring members, where phenyl and 5- and 6-membered hetaryl may have 1, 2, 3 or 4 substituents R^{a}, where R^{a} is selected from the group consisting of OH, SH, halogen, NO₂, NH₂, CN, COOH, CONH₂, C₁-C₈-alkyl, C₁-C₈-alkoxy, C₁-C₈-haloalkyl, C₁-C₈-haloalkoxy, C₁-C₈-alkylamino, di(C₁-C₈-alkyl)amino, C₁-C₈-alkylthio, C₁-C₈-haloalkylthio, C₁-C₈-alkylsulfinyl, C₁-C₈-haloalkylsulfinyl, C₁-C₈-alkylsulfonyl, C₁-C₈-haloalkylsulfonyl, C₃-C₈-cycloalkyl, phenyl, phenoxy and radicals of the formula C(=Z)R^{aa} in which Z is O, S, N(C₁-C₈-alkyl), N(C₁-C₈-alkoxy), N(C₃-C₈-alkenyloxy) or N(C₃-C₈-alkynyloxy) and R^{aa} is hydrogen, C₁-C₄-alkyl, C₁-C₄-alkoxy, NH₂, C₁-C₈-alkylamino or di(C₁-C₈-alkyl)amino;
or an agriculturally useful salt of a compound of the formula I.
except for a compound of the formula I in which R² is hydrogen or C₁-C₆-alkyl, R⁴ is phenyl which optionally carries 1, 2, 3 or 4 substituents R^{a} and Q is a fused saturated 5-, 6- or 7-membered carbocycle which is unsubstituted or has 1, 2, 3 or 4 C₁-C₄-alkyl groups as substituents or an agriculturally useful salt of this compound.

18. The compound according to claim 17 in which R¹ is selected from the group consisting of hydrogen, fluorine, chlorine, methyl, ethyl, methoxy, ethoxy, CF₃, CHF₂, OCF₃ and OCHF₂.

19. The compound according to claim 18 in which R¹ is hydrogen.

20. The compound according to claim 17 in which R² is selected from the group consisting of hydrogen, fluorine, chlorine, C₁-C₄-alkyl, methoxy, CF₃, CHF₂, OCF₃ and OCHF₂.

21. The compound according to claim 17 in which R² is hydrogen, methyl, methoxy or chlorine.

22. The compound according to claim 17 in which Q is one of the rings below: 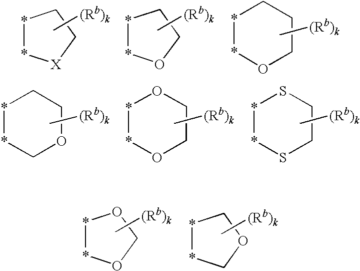
in which
* are the atoms of the pyrimidine ring to which Q is attached;
k is 0, 1, 2, 3 or 4;
R^{b} is C₁-C₄-alkyl; and
X is (CH₂)ₙ where n=1, 2 or 3 and where 1, 2, 3 or 4 of the hydrogen atoms in (CH₂)ₙ may be replaced by R^{b} if k≠0.

23. The compound according to claim 17 in which R³ is different from hydrogen.

24. The compound according to claim 23 in which R³ is fluorine, chlorine, C₁-C₄-alkyl or methoxy.

25. The compound according to claim 17 in which R⁴ is selected from the group consisting of 5-membered heteroaryl, which has 1, 2, 3 or 4 nitrogen atoms or 1 heteroatom selected from the group consisting of oxygen and sulfur and optionally 1, 2 or 3 nitrogen atoms as ring atoms, and 6-membered hetaryl which has 1, 2, 3 or 4 nitrogen atoms as ring members, where 5- and 6-membered hetaryl may have 1, 2, 3 or 4 substituents R^{a}.

26. The compound according to claim 25 in which R⁴ is selected from the group consisting of furyl, thienyl, pyridinyl and pyrimidinyl which are in each case unsubstituted or have 1, 2 or 3 substituents R^{a}.

27. The compound according to claim 25 in which the heteroaromatic radical R⁴ has at least one substituents and/or at least one ring member selected from the group consisting of O, S and N in the ortho-position to the point of attachment of R⁴ to the pyridine ring.

28. The compound according to claim 17 in which R^{a} is selected from the group consisting of halogen, C₁-C₄-alkyl, C₁-C₄-alkoxy, C₁-C₄-alkylcarbonyl, C₁-C₄-alkoxycarbonyl, and radicals of the formula C(═N—O—C₁-C₈-alkyl)R^{aa} in which R^{aa} is hydrogen or C₁-C₄-alkyl.

29. The use of a compound of the formula I according to claim 17 or of a salt thereof for controlling phytopathogenic fungi.

30. A crop protection composition comprising a solid or liquid carrier and a compound of the formula I according to claim 17 and/or a salt thereof.

31. Seed comprising at least one compound of the formula I according to claim 17 and/or a salt thereof.

32. A method for controlling phytopathogenic harmful fungi wherein the fungi, or the materials, plants, the soil or seed to be protected against fungal attack are/is treated with an effective amount of a compound of the formula I according to claim 17 or a salt thereof.
